SEQUENCE LISTING (12 sequences)

SEQ ID NO: 1 - DNA, 21 bp, Artificial Sequence
  Description of Artificial Sequence Synthetic primer
atttgaaagcgaagacagcgg

SEQ ID NO: 2 - DNA, 20 bp, Artificial Sequence
  Description of Artificial Sequence Synthetic primer
tggcgcccttctacctgtaa

SEQ ID NO: 3 - DNA, 22 bp, Artificial Sequence
  Description of Artificial Sequence Synthetic primer
gtggaagtgcagtttgatggag

SEQ ID NO: 4 - DNA, 22 bp, Artificial Sequence
  Description of Artificial Sequence Synthetic primer
tcaacttgaccctctaccacag

SEQ ID NO: 5 - DNA, 20 bp, Artificial Sequence
  Description of Artificial Sequence Synthetic primer
cagattacgctgctcgaggt

SEQ ID NO: 6 - DNA, 20 bp, Artificial Sequence
  Description of Artificial Sequence Synthetic primer
agacaaaagcagcggacgta

SEQ ID NO: 7 - DNA, 21 bp, Artificial Sequence
  Description of Artificial Sequence Synthetic primer
acagctgggtttctctacgtg

SEQ ID NO: 8 - DNA, 20 bp, Artificial Sequence
  Description of Artificial Sequence Synthetic primer
cagttactgcgacgtgaggt

SEQ ID NO: 9 - DNA, 20 bp, Artificial Sequence
  Description of Artificial Sequence Synthetic primer
cggtcgatgtatgtcttgtt

SEQ ID NO: 10 - DNA, 20 bp, Artificial Sequence
  Description of Artificial Sequence Synthetic primer
ctgggtttctctacgtgttc

SEQ ID NO: 11 - DNA, 19 bp, Artificial Sequence
  Description of Artificial Sequence Synthetic primer
ctccagggatcctgtgtcc

SEQ ID NO: 12 - DNA, 20 bp, Artificial Sequence
  Description of Artificial Sequence Synthetic primer
acaggaggtgggggttagac

(19) United States
(12) Patent Application Publication
(10) Pub. No.: US 2023/0279495 A1
(43) Pub. Date: Sep. 7, 2023

(54) MOLECULAR SIGNATURES OF RADIATION SENSITIVITY IN TUMORS, AND METHODS OF USE THEREOF

(71) Applicant: MEMORIAL SLOAN KETTERING CANCER CENTER, NEW YORK, NY (US)

(72) Inventors: Daniel Smith Higginson, New York, NY (US); Jonathan E. Leeman, Weymouth, MA (US)

(21) Appl. No.: 17/767,027

(22) PCT Filed: Oct. 7, 2020
(86) PCT No.: PCT/US20/54593

§ 371 Date: Apr. 6, 2022

Related U.S. Application Data
(60) Provisional application No. 62/911,917, filed on Oct. 7, 2019.

(51) Int. Cl. C12Q 1/6886; G16H 50/30; G16H 20/40

(52) U.S. Cl. CPC C12Q 1/6886; G16H 50/30; G16H 20/40; C12Q 2600/106; C12Q 2600/156; C12Q 2600/112

(57) ABSTRACT

The present invention relates to molecular signatures in genomic DNA that are associated with sensitivity of tumors, such as HPV-associated tumors, to treatment with radiation therapy. The present invention provides methods of analyzing tissue samples, or DNA obtained therefrom, to assess the presence of such molecular signatures, methods of predicting whether a tumor in a subject will be sensitive to treatment with radiation therapy based on the presence of such signatures, and methods of treating subjects with radiation therapy if such signatures are present.

### CROSS-REFERENCE TO RELATED APPLICATIONS

This application claims the benefit of priority of U.S. Provisional Patent Application No. 62/911,917 filed on Oct. 7, 2019, the content of which is hereby incorporated by reference in its entirety.

### STATEMENT REGARDING FEDERALLY SPONSORED RESEARCH

This invention was made with government support under grant number CA008748 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Oct. 7, 2020, is named MSKCC_043_WO1_SL.txt and is 3,018 bytes in size.

### INCORPORATION BY REFERENCE

For the purposes of only those jurisdictions that permit incorporation by reference, all of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Numbers in superscript or parentheses following text herein refer to the numbered references identified in the “Reference List” section of this patent application.

### BACKGROUND

Certain cancers, such as certain cancers that are associated with human papillomavirus (HPV) infection, are far more readily cured with radiation therapy than other cancers. However, the mechanism of this radiosensitivity is unknown. Across all of oncology, HPV-association is the only validated molecular biomarker of radiosensitivity. As such there is a need in the art for new and improved biomarkers of tumor radiosensitivity. The present invention addresses this need.

### SUMMARY OF THE INVENTION

Cancers associated with the human papillomavirus, including oropharyngeal, anal canal, cervical, and vulvar carcinomas, constitute about 4.5% of all solid tumors. They can be readily cured by therapeutic radiotherapy, which is the mainstay of treatment. HPV-associated cancers are more radiosensitive than HPV-negative cancers, but the mechanism of this radiosensitivity is unknown. Across all of oncology, HPV-association is the only validated molecular biomarker of radiosensitivity.

The present invention is based, in part, on a series of important new developments and discoveries relating to the mechanisms of radiosensitivity, which are described in more detail in the “Examples” section of this patent specification. To briefly summarize, it has now been discovered that the HPV16 E7 oncoprotein alters DNA double strand break repair pathways by promoting microhomology-mediated end-joining (MMEJ) and suppressing nonhomologous end-joining (NHEJ) without any suppression of repair by homologous recombination (HR). Furthermore, it has now also been discovered that, in subjects with HPV-positive head and neck squamous cell carcinomas, a subset of subjects having high levels of microhomology at breakpoints have improved disease free survival following radiation treatment as compared to subjects having low levels of microhomology. Building on these discoveries, the present invention provides a variety of new and improved methods for predicting sensitivity of subjects to treatment with radiation and for treating subjects with radiation, which are described in more detail in subsequent sections of this patent disclosure, including the Detailed Description, Examples, Drawings, and Claims sections.

### BRIEF DESCRIPTION OF THE DRAWINGS

FIG. 1 A-G. Human papillomavirus (HPV) associated head and neck cancers are associated with increased utilization of microhomology at deletion breakpoints without signatures of homologous recombination deficiency. (FIG. 1 A-E) The proportion of deletions with microhomology, large scale state transition (LST) scores and contribution of genomic Stratton signature 3 were measured in breast and ovarian cancers (n=1487) with or without bi-allelic BRCA1 or BRCA2 mutation, in HPV+ and HPV− head and neck cancers (n=510). HPV+ head and neck cancers demonstrated an increase in microhomology usage (FIG. 1 D) without an associated increase in LST score of signature 3 contribution (FIGS. 1 A and B). (FIG. 1 C) Schematic of Microhomology Mediated End Joining (MMEJ). (FIG. 1 E) BRCA mutations and HPV positivity were associated with an increase in microhomology length in these tumors. (FIG. 1 F) Whole exome sequencing data from all cancer types available in The Cancer Genome Atlas (TCGA) were analyzed for the presence of microhomology at mapped deletion sites, only deletions of 3+bp in length were considered. The proportion of deletions with 3+bp of microhomology across all cancer types is shown. (FIG. 1 G) Disease-free survival (DFS) of TCGA HPV+HNSCC cases classified by <50% of deletions containing MH and >=50% deletions containing MH. Assessable cases defined as those having a least 1 characterized deletion with MH. *represents a p-vale of <0.05, *** represents a p-value of <0.001.

FIG. 2 A-I. Human papillomavirus (HPV) E7 protein expression downregulates canonical non homologous end-joining (cNHEJ) and upregulates microhomology mediated end-joining (MMEJ). (FIG. 2 A-C) U20S cells with integrated double strand break repair reporter systems were transiently co transfected with Isce1 restriction enzyme and plasmids expressing individual HPV16 oncoproteins (E1, E2, E5, E6, E7). The percentage of GFP positive cells was measured 72 hours following transfection by flow cytometry (n=3+ repeats per condition). Cells expressing exogenous E7 oncoprotein showed an increase in HR and MMEJ but decreased cNHEJ. (FIG. 2 D) Stable transfection of U20S cells with the HPV16 E7 expression plasmid conferred significant reduction in pRB protein levels. On the right are survival curves based on clonogenic growth of U20S cells with integrated EJ5 reporter system transfected with empty vector (red) and HPV16-E7 (blue). Curves represent the mean of 4 experiments. (FIG. 2 E) Decrease in NHEJ activity measured by the percentage of GFP positive cells in U20S-EJ5 reporter cells with HPV16 E7 overexpression. (FIG. 2 F) HR activity measured in DR-GFP cells showed increase in HR activity with E7 expression but not in the cells expressing mutant E7 C24G protein defective in pRb binding. (FIG. 2 G) cNHEJ activity measured in U20S EJ5-GFP cells showed downregulation of cNHEJ activity with E7 expression but no difference in cells expressing E7 C24G. (FIG. 2 H) MMEJ activity measured in U20S EJ2-GFP cells showed upregulation of MMEJ activity with E7 expression but no significant difference in cells expressing E7 C24G. 3+ independent experiments were performed for each group. (FIG. 2 I) Relationship between E7 mRNA expression in TCGA HPV+ HNSCC cases and percentage of deletions with MH. Cases partitioned into quartiles of increasing E7 expression levels *represents a p-vale of <0.05, ** represents a p-value of <0.01, *** represents a p-value of <0.001.

FIG. 3 A-C. Effect of E7 on cNHEJ in the setting of IR mediated DNA damage. (FIG. 3 A) Representative immunofluorescent images of phospho-DNA-PKcs foci at 30 and 60 minutes after radiation in U20S cells stably transfected with an HPV16 E7 expressing vector or empty vector control. Green, phospho-DNA-PKcs foci; blue, DAPI staining. (FIG. 3 B) The number of pDNA-pk foci per cell detected was significantly decreased in the E7 expressing cell line compared to the EV expressing cell line. (FIG. 3 C) Data from 3 independent experiments represented as fold change in foci per cell shows significant fold change in cells expressing E7 after 60 and 240 minutes post IR. * represents a p-vale of <0.05, *** represents a p-value of <0.001.

FIG. 4. A-G. E7 expression shifts DNA double strand break repair pathway choice towards MMEJ. (FIG. 4 A) Schematic of DSBR-Seq Assay. The sequencing reads are classified into NHEJ (<5 bp deletion) in (FIG. 4 B), MMEJ (>5 bp deletion with at least 2 bp MH) in (FIG. 4 C) and HR (substitutions from HR donor) in (FIG. 4 D). The DNApk inhibitor Nu7741 was used at 1 μM (micromolar) concentration. (FIG. 4 E) cNHEJ measured as a fraction of mutant reads with deletion sizes less than 5 bp. (FIG. 4 F) E7 expression favors mutant reads with deletion sizes larger than 5 bp representing DSBs repaired by MMEJ. (FIG. 4 G) E7 expression results in lower deletion frequency of small deletions near the double strand break. Results represent the mean of 3 independent experiments+/−the standard error of the mean. * represents a p-vale of <0.05.

FIG. 5 A-C. Algorithm for determination of microhomology. Schematics of microhomology mediated end joining events that can result from either end of the microhomologous sequence are shown in (FIG. 5 A) and (FIG. 5 B). The logic framework utilized for identification of the presence and length of microhomology identified at a given breaksite is shown in (FIG. 5 C).

FIG. 6. Microhomology at deletion breakpoints and disease-free survival. Disease-free survival (DFS) Kaplan-Meier curves according to high MH (>=50% of deletions) or low MH (<50% of deletions) in patients who were treated with radiotherapy. DFS differences between groups were assessed by Log Rank testing.

FIG. 7 A-B. HPV associated cancers harbor alterations in double strand break repair factors. (FIG. 7 A) Expression of a subset of MMEJ factors are markedly increased in HPV+ tumors. (FIG. 7 B) Among all cancers in the TCGA, POLQ expression is highest in HPV+ head and neck cancers and cervical cancers, the vast majority of which are HPV associated (comparing HPV+HNSCC and cervical cancer to remaining cancers, p<0.001).

FIG. 8 A-C. Cassette reporter assays demonstrate E7 mediated pathway choice effects. (FIG. 8 A) RT-PCR confirmation of expression of each viral oncoprotein, including E7 E24G and E6/E7 in U2OS. (FIG. 8 B) Immunoblot of Rb expression when E7 wild type or E7 C24G is transiently expressed. (FIG. 8 C) Representative flow cytometry data demonstrating the influence of E7 on MMEJ (EJ2-GFP), NHEJ (EJ5-GFP), and HR (DR-GFP).

FIG. 9 A-C. Changes in DSB pathway choice are not associated with changes in cell cycle distribution. (FIG. 9 A) qPCR confirmation of elevated E7 expression following transfection with HPV16-E7 plasmid. (FIG. 9 B) Propidium iodide staining demonstrates no difference in cell cycle distribution between U2OS-empty vector and U2OS-E7 cell lines. (FIG. 9 C) MTT assay demonstrates no substantial difference in growth rate between U2OS-empty vector and U2OS-E7 cell lines.

FIG. 10 A-E. RB1 loss is associated with increased microhomology. (FIG. 10 A) Across all cancer types in the TCGA, the proportion of deletions among tumors with RB1 homozygous deletion is significantly higher than those without intact RB1. (FIG. 10 B) The mean length of microhomology is also increased among tumors with RB1 loss. (FIG. 10 C) A simulation found that RB1 loss enriches for microhomology compared to loss of 1000 random genes (p=0.029). (FIG. 10 D) Radar plot demonstrating proportion of deletions with microhomology by LST score demonstrates that BRCA mutation results in high LST and high MH, while RB1 loss or HPV-positivity results in low/moderate LST and high MH. (FIG. 10 E) Decreasing mRNA expression of RB1 across all TCGA tumors results in a graded increase in the presence of microhomology at deletion breakpoints.

FIG. 11. To evaluate for cancers that may have a pattern of high ID6 (ALT-EJ/MMEJ), but low/normal SBS3 we interrogated the PCAWG project, as described in Example 3. The results are plotted in graphical form in FIG. 11. HPV positive tumors (“HPV+ cervical SCC” and “HPV+HNSCC”) were shown to exhibit increased ID6 and low/normal SBS3. When correlating ID6 and SBS3, the HPV-associated cancers fall outside the confidence interval of the correlation (as shown by the dotted lines in the graph), showing that this is a statistically significant phenotype separate from that of other cancers.

### DETAILED DESCRIPTION OF THE INVENTION

The following Detailed Description of the invention is intended to be read in conjunction with all other sections of this patent disclosure, regardless of any heading or sub-heading titles. Furthermore, the various embodiments described below and in all other sections of this patent disclosure can be combined in various different ways, and all such combinations are intended to fall within the scope of the present invention.

### Definitions & Abbreviations

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Système International de Unites (SI) accepted form. Numeric ranges provided herein are inclusive of the numbers defining the range.

Where a numeric term is preceded by “about” or “approximately,” the term includes the stated number and values±10% of the stated number.

In all embodiments herein that refer to numeric value with the descriptor “about” (e.g. about 100 base pairs) alternate embodiments directed to the precise numeric value without the “about” descriptor are also contemplated and encompassed by the present invention.

Wherever embodiments are described with the language “comprising,” otherwise analogous embodiments described in terms of “consisting of” and/or “consisting essentially of” are also contemplated and encompassed by the present invention.

As used herein the phrase “substantially pure” or “substantially purified” (e.g. when used in reference to a DNA sample, e.g. a sample of genomic DNA, or whole genome genomic DNA, or whole exome genomic DNA) refers to a composition that is at least about 50%, more preferably at least about 75%, more preferably at least about 80%, more preferably at least about 85%, more preferably at least about 90%, more preferably at least about 95% pure, and most preferably at least about 99% pure. Thus, for example, a “substantially pure” sample of whole exome genomic DNA contains less than about 50%, preferably less than about 25%, preferably less than about 20%, preferably less than about 15%, preferably less than about 10%, preferably less than about 5%, and most preferably less than about 1% of non-exomic DNA.

Numbers in parentheses or superscript following text in this patent disclosure (particularly in the Background and Examples sections) refer to the numbered references provided in the “Reference List” section at the end of this patent disclosure.

Other abbreviations and definitions may be provided elsewhere in this patent specification, or may be well known in the art.

### Additional Detailed Description of the Invention

The present invention provides a variety of new and improved methods for predicting the sensitivity of tumors to treatment with radiation and for treating subjects with radiation.

Accordingly, in some embodiments the present invention provides methods of predicting the radiation sensitivity of a tumor (such as an HPV-positive tumor) in a subject, such methods comprising analyzing the sequence of a genomic DNA sample from a tumor (e.g. an HPV-positive tumor) in a subject to assess microhomology at DNA break-sites, wherein the existence of high levels of microhomology at break-sites indicates that the tumor in the subject is likely to be sensitive to treatment with radiation. In some embodiments such methods further comprise analyzing the sequence of the genomic DNA sample to asses DNA repair by homologous recombination, wherein the existence of both: (a) high levels of microhomology at break-sites and (b) normal or high levels of DNA repair by homologous recombination indicates that the tumor in the subject is likely to be sensitive to treatment with radiation. In some embodiments such methods further comprise analyzing the sequence of the genomic DNA to assess DNA repair by non-homologous end joining (NHEJ), wherein the existence of each of: (a) high levels of microhomology at break-sites, (b) normal or high levels of DNA repair by homologous recombination, and (c) low levels of DNA repair by NHEJ, indicates that the tumor in the subject is likely to be sensitive to treatment with radiation. In some such embodiments the genomic DNA sample is a whole genome genomic DNA sample. In some such embodiments the genomic DNA sample is a whole exome genomic DNA sample.

In some embodiments, the present invention also provides various methods of treatment. For example, in some embodiments, the present invention provides methods of treating a tumor (e.g. an HPV-positive tumor) in a subject in need thereof, the methods comprising predicting the radiation sensitivity of tumor (e.g. an HPV-positive tumor) in a subject using a method as described above, or elsewhere herein, and then, if the results of such method indicate that the tumor in the subject is likely to be sensitive to treatment with radiation, subsequently treating the tumor in the subject with radiation.

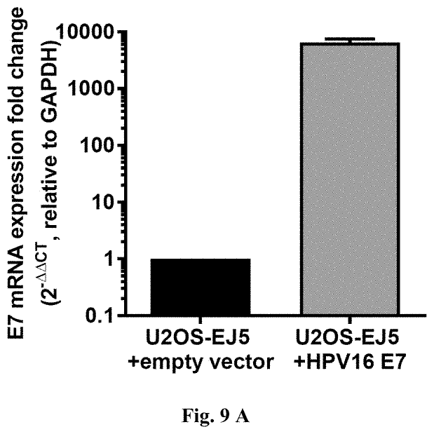

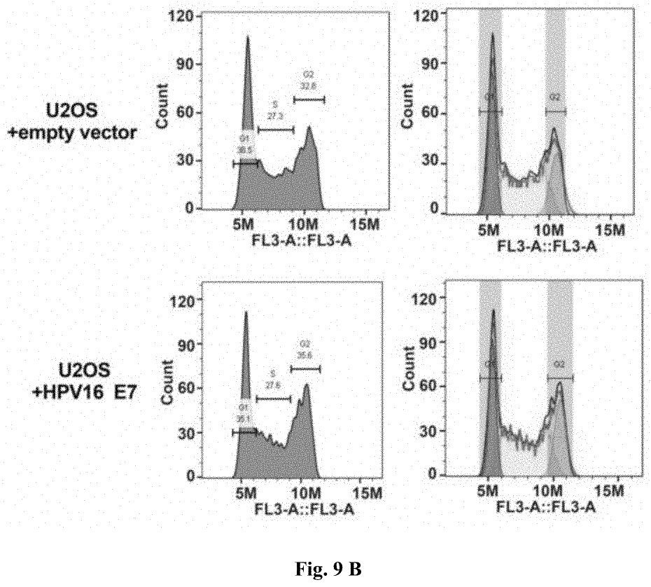

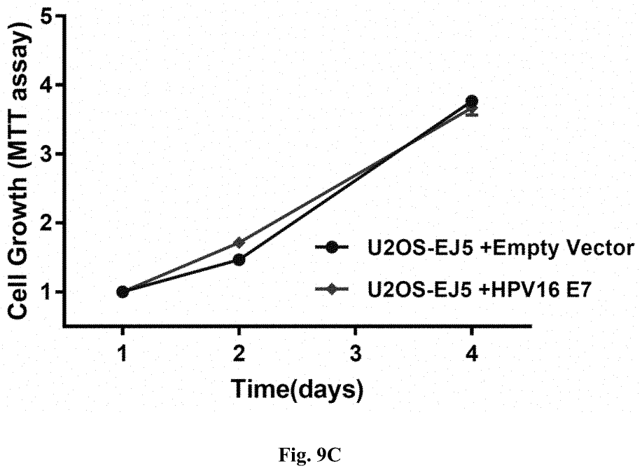

In each of the embodiments described above and elsewhere herein that involve an assessment of microhomology at DNA break-sites, the assessment can be performed using any suitable method known in the art or provided herein. Typically, the existence of microhomology at DNA break-sites is detected by analyzing the sequence spanning deletions/breaksites in the genomic DNA and determining whether there is microhomology in the regions spanning such deletions/breaksites. One such method employs the algorithm provided in FIG. 9. In one embodiment microhomology at DNA break-sites is assessed by: (a) locating the site of a specific deletion in the genomic DNA sample (i.e. a “breaksite”) by comparing the sequence of the genomic DNA sample to a control DNA sequence that does not have the deletion, and (b) starting at the breaksite, comparing the sequence of base pairs immediately upstream and downstream of the DNA breaksite to the sequence of the control DNA sequence that has been deleted, and (c) determining how many base pairs immediately upstream and downstream of the DNA breaksite are identical to base pairs of the deleted sequence, wherein if one or more base pairs is identical to base pairs of the deleted sequence, then there is microhomology at the breaksite. In another embodiment microhomology at DNA break-sites is assessed by: (a) locating a deletion site/breaksite in the genomic DNA sample (e.g. by comparing the sequence of the genomic DNA sample to a control DNA sequence that does not have the deletion), and (b) labeling/assigning basepairs immediately upstream of the deleted sequence as positions 0, −1, −2, and so on in sequence 5′ to the deleted site, (c) labeling/assigning the last basepair of the deletion as +L and basepairs downstream from the deletion as L+1, L+2, and so on in sequence 3′ to the deletion, and then executing the following algorithm:
1. compare position 0 and position L (test 1),
2. If position 0 and position L are identical, then −x and L−x are compared and so in 1 bp increments (test 2). If identical, then −x and L−x are compared in 1 bp increments until the base pairs are no longer identical,
3. If 0 and L are not identical, then position +1 and (L+1) are compared (test 3). If identical then (+2 and L+2) and compared and so on in 1 bp increments until these comparison bases are no longer identical (test4).
4. If test 1 and test 3 are negative, the number of bp of MH is defined as 0.
5. If test 1 is positive and test 3 is negative, microhomology is defined as (−x) to 0, inclusive.
6. If test 1 is negative and test 3 is positive, then MH is defined as the larger of [(−x) to 0, inclusive] OR [(+1) to (+y), inclusive].
7. If both test 1 and 3 are positive, then MH is defined as the larger of [(−x) to 0, inclusive] OR [(+1) to (+y), inclusive]

Other suitable methods of assessing microhomology at DNA break-sites include those that use one or more of the algorithms provided at http://www.github.com/xqrongm/delmh and/or the HRDetect microhomology algorithm—all of which are hereby incorporated by reference. In some embodiments microhomology at DNA break-sites is assessed by determining the percentage of deletions in the genome (or in the portion of the genome that is analyzed, e.g the whole exome) that are flanked on either side by micro-homologous sequences, e.g. micro-homologous sequences of at least 3 base pairs in length. In some embodiments, if more than 50% of the deletions in the genome (or in the portion of the genome that is analyzed) are flanked on either side by micro-homologous sequences (e.g. of at least 3 base pairs in length), then it is considered that there are “high” levels of microhomology at break-sites. Other definitions of “high” levels of microhomology are provided below and/or are known in the art. Importantly, the data presented in the Examples section of this patent disclosure shows that when more than 50% of the deletions in the genome (or in the portion of the genome that is analyzed) are flanked on either side by micro-homologous sequences of at least 3 base pairs in length, then the subject has a greater than 50% chance of surviving disease free for 3 years after radiation treatment, whereas if less than 50% of the deletions in the genome are flanked on either side by micro-homologous sequences of at least 3 base pairs in length then the subject has a less than 25% chance of surviving disease free after radiation treatment.

In each of the embodiments described above and elsewhere herein that involve an assessment of DNA repair by homologous recombination, the assessment can be performed using any suitable method known in the art or provided herein. One such method is based on assessing the presence and/or amount of large-scale state transitions (LSTs) or LST scores. Another such method is based on assessing the presence and/or amount of loss of heterozygosity (LOH) or LOH scores. Another such method is based on assessing the presence and/or amount of telomeric allelic imbalance (TAI) or TAI scores. Another such method is based on assessing a combination of the presence and/or amount of LSTs (or LST scores), LOH (or LOH scores), and TAI (or TAI scores). Such a combined method is employed by the commercially available myChoice HRD system provided by Myriad Genetics. Another such method is based on assessing the presence and/or amount of Signature 3 rearrangement signatures or Signature 3 scores. Such a method is employed by the commercially available HRDetect system provided by the Wellcome Sanger Trust. Additional methods of assessing LSTs or LST scores are described in Abkevich et al. (2012) (Patterns of genomic loss of heterozygosity predict homologous recombination repair defects in epithelial ovarian cancer. Br J Cancer 107(10):1776-1782), Popova et al. (2012) (Ploidy and large-scale genomic instability consistently identify basal-like breast carcinomas with BRCA1/2 inactivation. Cancer Res 72(21):5454-5462), Riaz et al. (2017) (Pan-cancer analysis of bi-allelic alterations in homologous recombination DNA repair genes. Nat Commun 8(1):857), Mutter et al. (2017) (Bi-allelic alterations in DNA repair genes underpin homologous recombination DNA repair defects in breast cancer. J Pathol 242(2):165-177), and Manie et al. (2016) (Genomic hallmarks of homologous recombination deficiency in invasive breast carcinomas. Int J Cancer 138(4):891-900)—the contents of each of which are hereby incorporated by reference. Similarly, additional methods for assessing Signature 3 rearrangement signatures or Signature 3 scores are described in Alexandrov et al. (2013) (Signatures of mutational processes in human cancer. Nature 500(7463):415-421) and Nik-Zainal S, et al. (2016) (Landscape of somatic mutations in 560 breast cancer whole-genome sequences. Nature 534(7605):47-54)—the contents of each of which are hereby incorporated by reference. Another such method involves assessment of ID6 signatures (which have been shown to be associated with cancers with biallelic inactivation of the homologous recombination genes BRCA1 and BRCA2). The ID6 signature is associated with deletions >5 bp and >2 bp of microhomology used in repairing the deletion. Another such method involves assessment of ID8 signatures (which have been shown to be associated with cancers with biallelic inactivation of the homologous recombination genes BRCA1 and BRCA2). Furthermore, additional methods for assessment of DNA repair by homologous recombination are provided by the following references and patents/patent applications: US20190139625A1; US20190130997A1; US20190119759A1; U.S. Ser. No. 10/400,287B2; U.S. Ser. No. 10/400,287B2; Zhao et al. Clin Cancer Res; 23(24) Dec. 15, 2017; Telli et al., Clin Cancer Res; 22(15) Aug. 1, 2016; Davies et al., Nature Medicine, 23 (4), April 2017; Popova et al., Cancer Res; 72(21) Nov. 1, 2012—each of which is hereby incorporated by reference herein. Other suitable methods for assessing genetic markers of homologous recombination are known in the art and can be used in conjunction with the present invention.

In each of the embodiments described above and elsewhere herein that involve an assessment of DNA repair by non-homologous end joining, the assessment can be performed using any suitable method known in the art or provided herein.

Each of the embodiments described above and elsewhere herein may also comprise an initial step of obtaining a tissue sample from the subject. Similarly, each of the embodiments described above and elsewhere herein may also comprise an initial step of obtaining a genomic DNA sample from the subject. Similarly, each of the embodiments described above and elsewhere herein may also comprise an initial step of obtaining a whole genome genomic DNA sample from the subject. Similarly, each of the embodiments described above and elsewhere herein may also comprise an initial step of obtaining a whole exome genomic DNA sample from the subject. Some of the embodiments described above and elsewhere herein may also comprise initial steps of: (a) obtaining a sample of genomic DNA from a tumor in a subject, (b) fragmenting the genomic DNA, (c) contacting the fragmented genomic DNA with one or more probes that selectively hybridize to exomic DNA, (d) substantially purifying the fragmented DNA that is selectively bound by the probes to obtain a substantially pure sample of whole exome genomic DNA, and (e) using the substantially pure sample of whole exome genomic DNA in place of the sample of genomic DNA in all subsequent method steps.

Each of the embodiments described above and elsewhere herein may also comprise performing a test to determine if the tumor in the subject is HPV-positive, and/or to determine if the tumor in the subject expresses HPV E7, and/or to determine the levels of POLQ expression in the subject's tumor.

In each of the embodiments described above and elsewhere herein, the sequence of the genomic DNA may be determined and/or analyzed using any suitable method known in the art. In some embodiments the sequence of the genomic DNA may be determined using a DNA sequencing method, such as a next generation sequencing (NGS) method, for example an Illumina next generation sequencing (NGS) method.

In each of the embodiments described above and elsewhere herein, the sequence of the genomic DNA may be compared to that of a suitable control genomic DNA sequence.

In some of the embodiments described above and elsewhere herein, the subject may have an HPV-positive squamous cell carcinoma (SCC).

In some of the embodiments described above and elsewhere herein, the subject may have an HPV-positive squamous cell carcinoma of the head and/or neck (HNSCC).

In some of the embodiments described above and elsewhere herein, the subject may have an HPV-positive squamous cell carcinoma (SCC) of the oropharynx, cervix, vulva or anal canal.

In some of the embodiments described above and elsewhere herein, the subject may have an HPV-16, 18, 31, 33, 35, 39, 45, 51, 52, 56, 58 or 59-positive tumor.

In each of the embodiments herein that refer to a control sample, any suitable control sample may be used—as can readily be determined by those of skill in the art. For example, in some embodiments the control sample may be a tissue sample or DNA sample from a radiation-resistant tumor, e.g. of the same tumor type as the subject's tumor. This would be a negative control. Conversely, in some embodiments the control sample may be a tissue sample or DNA sample from a radiation-sensitive tumor, e.g. of the same tumor type as the subject's tumor. This would be a positive control. In cases where the subject has an HPV-positive tumor, the control sample may be a tissue samples or DNA sample from an HPV-negative tumor (i.e. a negative control). Conversely, the control sample may be a tissue sample or DNA sample from an HPV-positive tumor (i.e. a positive control). In some embodiments the control sample may be a tissue sample or DNA sample from non-tumor tissue. This would be a negative control. In some embodiments a control sample can be from the subject himself or herself, while in other embodiments the control sample can be from a different subject/individual. In some embodiments control samples, or genomic DNA sequences from suitable control samples, can be those in any suitable database or repository. For example, in some cases the control may be a genomic DNA sequence in a genome sequence database or repository—i.e. without any need for physically obtaining a control sample from a subject.

In each of the embodiments herein that refer to a genetic signature or a score relating to such a genetic signature (e.g. an LST score, a TAI score, a TAI score, a signature 3 score, an ID6 score, and ID8 socre) as being “high” or “low” the determination of what constitutes high or low is made with respect to what would be considered a “normal” level—as understood by those of ordinary skill in the art. For example, in some embodiments a “high” level is a level that is about 10%, or about 20%, or about 30%, or about 40%, or about 50%, or about 60%, or about 70%, or about 80%, or about 90% or about 100% or about 150%, or about 200% or about 250% or more higher than the “normal” level—e.g. the corresponding level in a suitable normal control sample such as, for example, in a non-tumor cell, or in a “wild-type” cell, or in a genomic DNA sequence from a non-tumor cell or a “wild-type” cell in a suitable genetic sequence database or repository.

While the main embodiments of the present invention are summarized above, additional embodiments and additional details are provided and described in the Brief Description of the Drawings, Examples, Claims, and Drawings/Figures sections of this patent application. Furthermore, it should be understood that variations and combinations of each of the embodiments described above and elsewhere herein are contemplated and are intended to fall within the scope of the present invention.

### EXAMPLES

The invention is further described by the following non-limiting “Examples,” as well as the Figures referred to therein and the descriptions of such Figures provided above.

### Example 1

Note that the references cited in Example 2 are provided in Reference List No. 1

Squamous cell carcinomas (SCCs) arising from aerodigestive or anogenital epithelium that are associated with the human papillomavirus (HPV) are far more readily cured with radiation therapy than HPV-negative SCCs. The mechanism behind this increased radiosensitivity has been proposed to be secondary to defects in DNA repair, though the specific repair pathways that are disrupted have not been elucidated. To gain insight into this important biomarker of radiosensitivity, we first examined genomic patterns reflective of defects in DNA double strand break repair, comparing HPV-associated and HPV-negative head and neck cancers (HNSCC). Compared to HPV-negative HNSCC genomes, HPV+ cases demonstrated a marked increase in the proportion of deletions with flanking microhomology, a signature associated with a backup, error-prone double strand break repair pathway known as microhomology-mediated endjoining (MMEJ). Then, using three different methodologies to comprehensively profile double strand break repair pathways in isogenic paired cell lines, we demonstrate that the HPV16 E7 oncoprotein suppresses canonical non-homologous end-joining (NHEJ) and promotes error-prone MMEJ, providing a mechanistic rationale for the clinical radiosensitivity of these cancers.

### Introduction

The human papillomavirus (HPV) is an oncogenic virus that has been long linked to multiple anogenital cancers including cervical cancer, anal cancer, penile cancer and vulvar cancer(1). In the last two decades, it has become clear that HPV is responsible for a rapidly increasing proportion of head and neck squamous cell carcinomas (HNSCC), specifically oropharyngeal cancers (OPC) (2). The prognosis of HPV-associated OPC is markedly improved compared to HPV-negative OPC(3, 4), largely due to an improved response to chemoradiation. However, the underlying mechanisms responsible for this marked sensitivity to ionizing radiation (IR) and DNA damaging agents remain unknown.

Preclinical studies have demonstrated that HPV+HNSCC cell lines are intrinsically more radiosensitive than their HPV− counterparts, suggesting that HPV infection plays a direct role in radiosensitivity(5, 6). Microhomology mediated end joining (MMEJ) is a DSB repair pathway that may play a critical role in radiosensitivity(7, 8) as it is highly error prone and may lead to lethal repair events. MMEJ is thought to serve as a backup pathway that is activated by defects that occur in the major cNHEJ or HR pathways(9), as has been demonstrated in HR-deficient breast and ovarian tumors(10). While multiple lines of evidence suggest that HPV oncoproteins interfere with the DNA damage response, the DSB repair pathways that are affected have yet to be clarified.

Defects in cNHEJ have been identified in multiple human cancers and are known to be highly radiosensitizing(11-16). RB1, the primary target of HPV E7, has been reported to play a direct role in cNHEJ through recruitment of XRCC5/6 (Ku70/80) to the sites of double strand breaks, suggesting a potential role for E7 in dysregulation of DSB repair(17). Furthermore, disruption of cNHEJ often results in a reciprocal increase in MMEJ and genomic sites of HPV viral integration in cervical and oropharyngeal cancers have been found to be highly enriched for microhomology, the defining hallmark of MMEJ, suggesting an association between HPV and MMEJ(18, 19).

In the current study, we demonstrate that HPV-associated tumors reflect genomic scars associated with frequent use of MMEJ. Furthermore, we find that HPV16-E7 expression alone promotes radiosensitivity and is primarily responsible for alterations in DSB repair pathway choice that underly these changes.

### Results

HPV-Associated HNSCC Genomes Feature Deletions with Short Stretches of Microhomology.

In the absence of effective HR, there is increased use of backup double strand break repair pathways that lead to increased translocations and deletions over the course of carcinogenesis, which can be quantified and recognized in tumor genomes using a computational algorithm called Large-scale State Transitions (LST)(20, 21). The LST score quantifies the number of genomic alterations consisting of a rejoining repair event of two double strand breaks at least 10 Mb apart in the genome. High LST scores in cancer genomes are associated with germline or somatic mutations in BRCA1 or BRCA2 as well as other HR genes.(22-24) In addition, a characteristic base substitution pattern known as Signature 3 is also strongly associated with BRCA1/2 mutations and HR deficiency(25, 26). The signature 3 pattern was empirically discovered and is readily distinguished from other base substitution patterns from aging, tobacco smoke, and the effect of APOBEC enzymes. Elevated LST and Signature 3 characterize tumors deficient in HR, including cancers with mutations in HR genes other than BRCA1/2 or cancers with non-genetic mechanisms of HR inactivation such as hypermethylation of the BRCA1 promoter(22, 23).

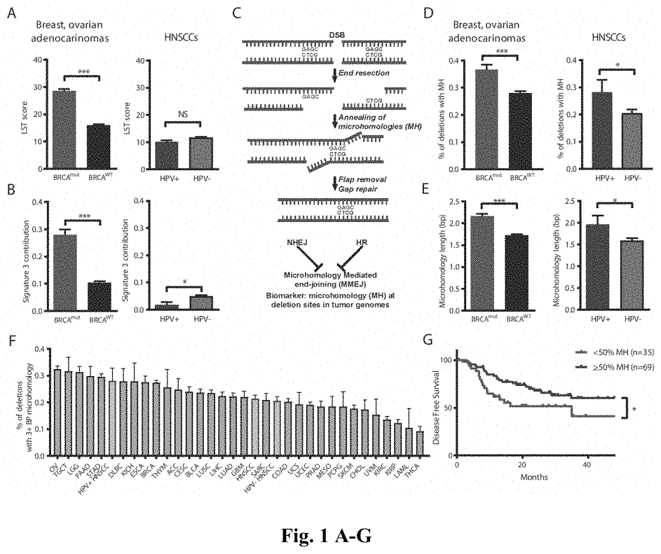

To characterize possible effects of HPV infection on DNA repair in human HNSCCs, we first profiled LST and Signature 3 scores in human cancer genomes sequenced as part of the Cancer Genome Atlas (TCGA) project, comparing HPV+ and HPV− HNSCCs (FIG. 1 A, B). Cancers with bi-allelic BRCA1/2 mutations (BRCA^{mut}) controls in breast and ovarian cancers demonstrate higher LST and Signature 3 scores, as expected, whereas HPV+HNSCCs exhibit low LST scores and no difference from HPV− HNSCCs. Both HPV+ and HPV− HNSCCs also have very low Signature 3 scores and in fact HPV− HNSCCs exhibit higher mean Signature 3 scores. These data indicate that an HR deficiency is unlikely to explain the inherent radiosensitivity of HPV+HNSCCs.

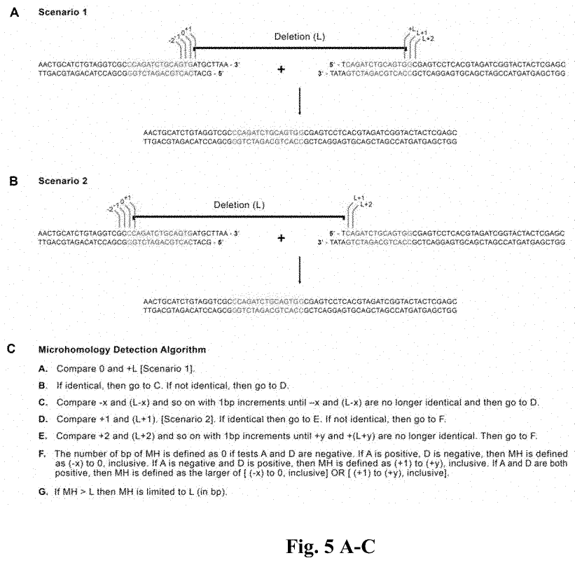

An additional potential biomarker of HR deficiency is the predominance of microhomologous sequences on either side of small deletions (FIG. 1 C). MMEJ is viewed as a backup end-joining pathway for both HR and NHEJ. In order to assess the role of MMEJ across multiple cancer types, we interrogated the Cancer Genome Atlas (TCGA) whole exome sequencing datasets across 32 cancer types (n=10,271 tumors, FIG. 5). Individual deletion breakpoints called by the TCGA project were assessed for the presence of microhomology flanking the breaksite, suggesting a DSB repair event mediated by MMEJ. Deletions with surrounding microhomology lengths of ≥3 base pairs were considered to be MMEJ associated, as it has been previously demonstrated that the central MMEJ factor polymerase theta (POLQ) mediates repair breakpoints with 3+ base pairs of microhomology(27). Ovarian cancers were found to have the highest proportion of microhomology associated deletions (32.5%, FIG. 1 F), consistent with a high percentage of ovarian cancers with HR defects (HRD)(28). Overall, HNSCCs were found to have an intermediate amount of microhomology (21.3% of deletions), though HPV+HNSCCs demonstrated a higher proportion of deletions associated with 3+bp of MH than HPV− HNC (HPV+n=77 28.1% vs HPV− n=433 20.6%, p=0.04) shown in FIG. 1 D. The mean number of base pairs of microhomology associated with breaksites is likewise increased in HPV+HNSCC compared to HPV− HNSCC (FIG. 1 E). Taken together, this genomic analysis suggests an increased utilization of MMEJ in HPV+HNSCCs without the genomic hallmarks of HR deficiency (high LST and Signature 3).

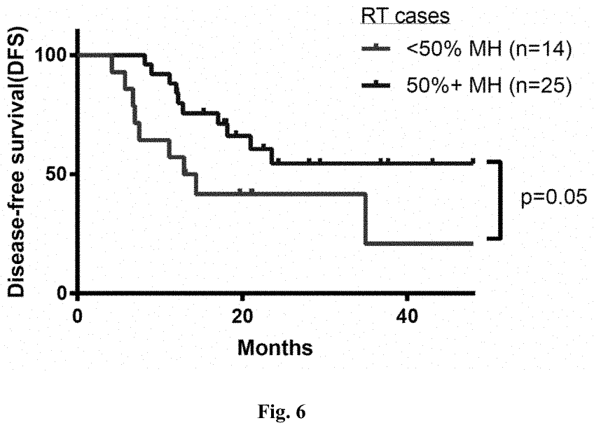

Since microhomology (MH) at deletion sites is associated with radiosensitive HPV+ SCCs, we then examined whether MH associates with clinical outcomes (FIG. 1 G, FIG. 6 and Tables 1-3). Assessment of clinical outcomes in TCGA HPV+HNSCC cases identified improved disease free survival (DFS) in cases where >50% of deletions were associated with MH (3-year DFS: 60.1% in MH≥50% group, 41.2% in MH<50% group, p=0.04, including only cases assessable with at least 1 deletion associated with MH). Furthermore, this effect was found to persist when including only patients who were treated with radiotherapy (3-year DFS: 54.5% in MH≥50% group, 20.8% in MH<50% group, p=0.04, suggesting that a high proportion of deletions with associated MH is linked to radiosensitivity (FIG. 6 and Tables 1-3).

TABLE 1

| | | | | | TCGA HNSCC | | |
|---|---|---|---|---|---|---|---|
| | | All TCGA HNSCC cases | | | cases receiving RT | | |
| | | Low MH | High MH | | Low MH | High MH | |
| | | (N = 35) | (N = 69) | p | (N = 14) | (N = 25) | p |
| Sex | M | 30 | 52 | 0.22 | 12 | 16 | 0.15 |
| | F | 5 | 17 | | 2 | 9 | |
| Race | White | 28 | 59 | 0.13 | 12 | 20 | 0.91 |
| | Black or AA | 7 | 6 | | 2 | 3 | |
| | Asian | 0 | 1 | | 0 | 0 | |
| | Unknown | 0 | 3 | | 0 | 2 | |
| Median age, yrs | | 60 | 62 | 0.63 | 60 | 59 | 0.81 |
| (range) | | (19-88) | (30-87) | | (42-69) | (47-82) | |
| Median follow- | | 20.2 | 25.0 | 0.30 | 18.8 | 22.4 | 0.18 |
| up, mo (range) | | (3.5-107.4) | (0.36-140.67) | | (6.4-51.1) | (11.3-72.5) | |
| Stage (AJCC | I | 1 | 1 | 0.99 | 0 | 1 | 0.93 |
| 7th) | II | 5 | 10 | | 0 | 2 | |
| | III | 9 | 17 | | 3 | 5 | |
| | IVA | 18 | 37 | | 11 | 17 | |
| | IVB | 1 | 2 | | 0 | 0 | |
| | Unknown | 1 | 2 | | 0 | 0 | |
| T-stage | 1 | 2 | 5 | 0.95 | 1 | 2 | 0.79 |
| | 2 | 6 | 15 | | 0 | 5 | |
| | 3 | 11 | 21 | | 5 | 5 | |
| | 4 | 15 | 26 | | 8 | 13 | |
| | X | 1 | 1 | | 0 | 0 | |
| | Unknown | 0 | 1 | | 0 | 0 | |
| N-stage | 0 | 17 | 34 | 0.96 | 4 | 13 | 0.36 |
| | 1 | 6 | 10 | | 3 | 4 | |
| | 2 | 11 | 22 | | 7 | 8 | |
| | 3 | 0 | 1 | | 0 | 0 | |
| | X | 1 | 1 | | 0 | 0 | |
| | unknown | 0 | 1 | | 0 | 0 | |
| Chemotherapy | Yes | 7 | 12 | 0.82 | 7 | 12 | 0.89 |
| | No | 10 | 17 | | 6 | 12 | |
| | Unknown | 18 | 40 | | 1 | 1 | |
| Radiation | Yes | 14 | 25 | 0.66 | N/A | N/A | |
| | No | 4 | 5 | | N/A | N/A | |
| | Unknown | 17 | 39 | | N/A | N/A | |
| HPV status | Pos | 1 | 11 | 0.048 | 1 | 5 | 0.29 |
| | Neg | 34 | 58 | | 13 | 20 | |
| Smoking History | Yes | 25 | 38 | 0.11 | 11 | 15 | 0.24 |
| | No | 10 | 31 | | 3 | 10 | |
| Primary site | Oropharynx | 3 | 12 | 0.47 | 2 | 6 | 0.17 |
| | Oral cavity | 22 | 40 | | 10 | 10 | |
| | Larynx/ | 10 | 17 | | 2 | 9 | |
| | hypopharynx | | | | | | |

Table 1 provides patient characteristics of all assessable TCGA HNSCC cases and those cases receiving radiotherapy. Statistical differences between the groups were tested using Chi-squared and t-test.

TABLE 2

| Univariate | HR | CI | P |
|---|---|---|---|
| HPV positive status | 0.15 | (0.2-1.06) | 0.057 |
| <50% of deletions | 0.53 | (0.28-0.99) | 0.046 |
| with ≥3 bp of | | | |
| microhomology | | | |

TABLE 3

| Univariate | HR | CI | P |
|---|---|---|---|
| HPV positive status | 0.23 | (0.31-1.74) | 0.16 |
| <50% of deletions | 0.42 | (0.17-1.04) | 0.06 |
| with ≥3 bp of | | | |
| microhomology | | | |

Tables 2 and 3 relate to FIG. 6 and the data therein regarding Disease-free survival 3(DFS) according to high MH (>=50% of deletions) or low MH (<50% of deletions) in patients who were treated with radiotherapy. DFS differences between groups were assessed by Log Rank testing in FIG. 6 and Cox regression analysis in Table 2 and Table 3.

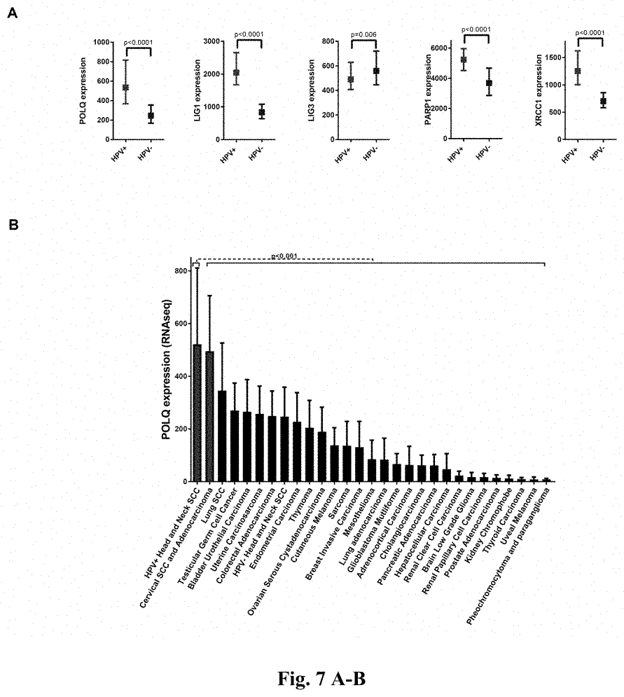

The high MH phenotype implies increased use of MMEJ in HPV+HNSCCs. However, the expression of the central MMEJ factor POLQ is thought to vary widely across tissue types(29, 30) and the expression of POLQ is increased in some cancers requiring backup MMEJ(10). Thus, we examined expression levels of POLQ and other MMEJ factors in the TCGA RNAseq datasets and expression of MMEJ factors is robust in HPV+HNSCCs, indicating that MMEJ may be an active pathway (FIG. 7).

### HPV16 E7 Increases Usage of Microhomology-Mediated End-Joining at DSBs.

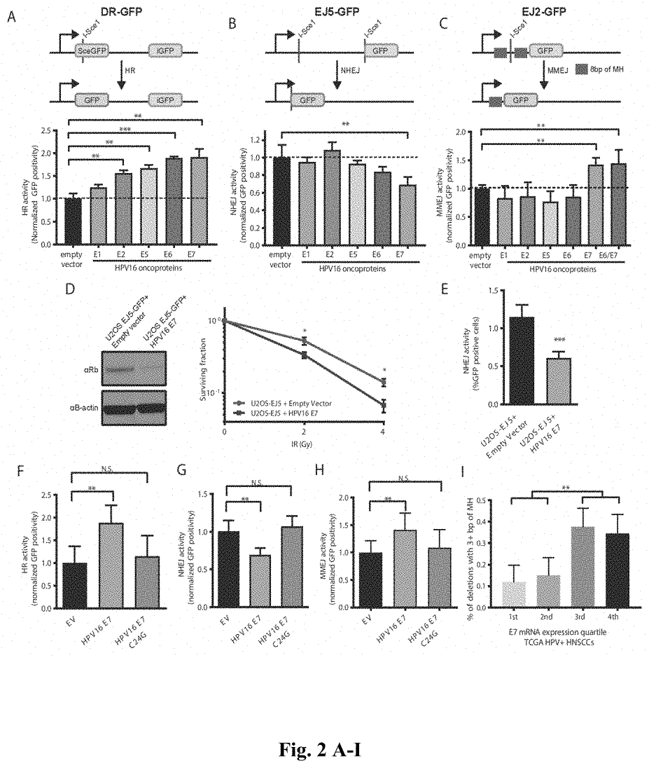

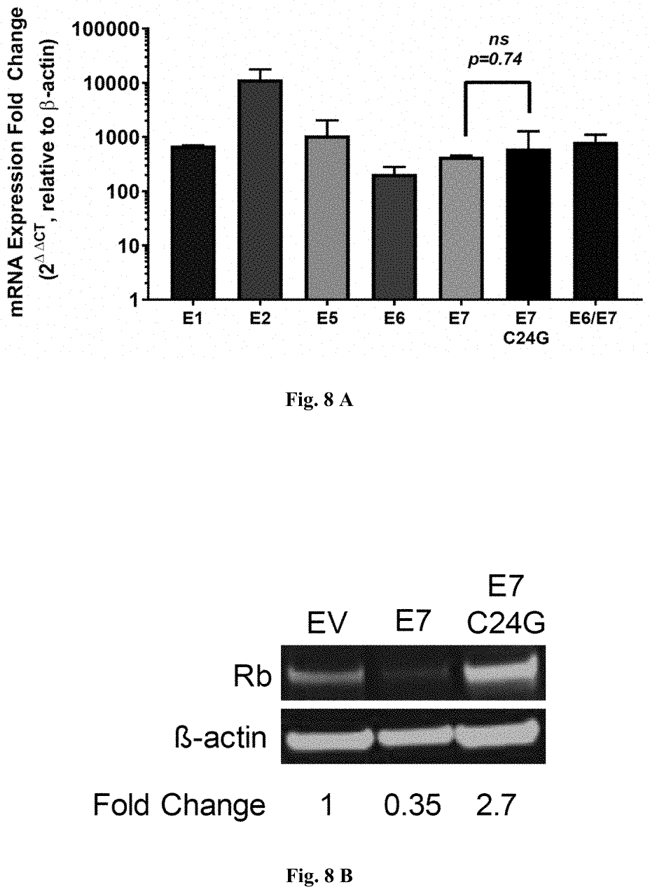

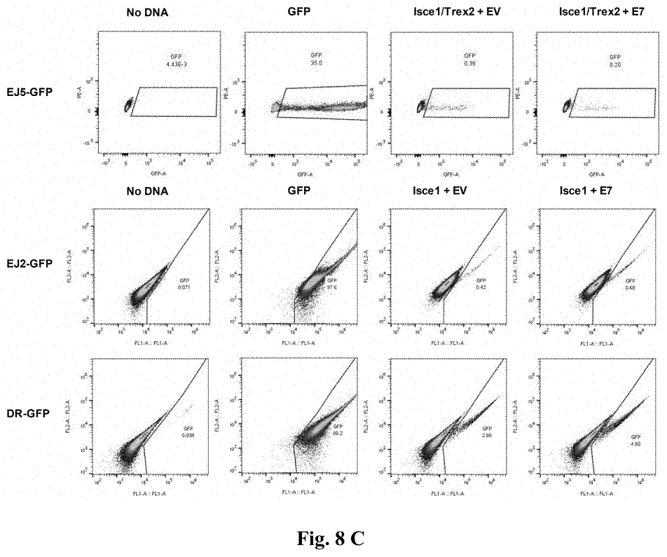

As our genomic analysis identified an increase in MMEJ utilization, we then directly assessed the effect of HPV oncoprotein expression on DSB repair pathway choice. We employed previously described U2OS reporter cell lines designed to measure MMEJ (EJ2-GFP(31)), cNHEJ (EJ5-GFP) and HR (DR-GFP) after a site-directed DSB is created by an expressed endonuclease I-SceI. We first measured the effects of each oncoprotein using expression plasmids for HPV oncoproteins that were transiently transfected into each reporter cell line, including E1, E2, E5, E6, E7, and the percentage of GFP positive cells was measured 72 hours later by flow cytometry (FIG. 2 and FIG. 8). Compared to an empty vector control, only expression of HPV16 E7 resulted in a 42% increase in MMEJ activity (FIG. 2 C, p=0.005) and a 31% decrease in NHEJ activity (FIG. 2 B, p=0.002). Multiple HPV16 oncoproteins (E2, E5, E6, E7) upregulated homologous recombination (FIG. 2 A). A similar increase in MMEJ was observed when both E6 and E7 were expressed (FIG. 2 C). Because defects in NHEJ are most likely to explain radiosensitivity, we focused on the effect of E7 on repair pathway choice.

Using a stably transfected U2OS clone expressing E7, we verified E7 expression with qPCR, demonstrated decreased Rb levels and increased radiosensitivity (FIG. 2 D) compared to a U2OS clone transfected with an empty vector. Cell cycle distribution and growth kinetics were identical with or without E7 (FIG. 9). These control experiments verify that E7 in our model sequesters and degrades Rb, but does not alter cell cycle kinetics relative to the U2OS host. Using this fully characterized clone, we confirmed that E7 suppresses NHEJ while increasing MMEJ and HR (FIG. 2. E-H). We then found that the E7 effect on these repair pathway choices is dependent upon a residue within the E7 LXCXE domain that is required for binding to Rb (FIG. 2 F-H). The E7 C24G mutant with defective Rb binding LXCXE domain was chosen as it inactivates Rb as well as associated pocket proteins p107/p130.(32)

To further evaluate whether E7 is the likely driver of increased MMEJ, we again evaluated the HPV+HNSCC cancer genomes and partitioned the cases into quartiles of low to high E7 expression by RNAseq(33). This found a dose response relationship between E7 expression and the MH phenotype (FIG. 2 I).

### HPV16 E7 Suppresses Formation of Phospho-DNApkcs Irradiation-Induced Foci.

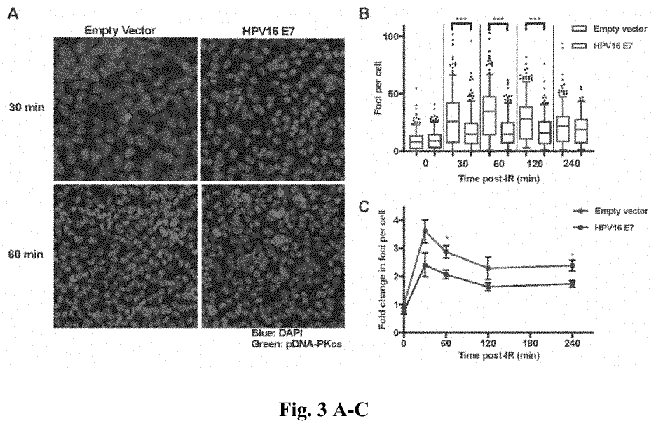

Following recognition of a DSB by Ku70/80, the DNA-dependent protein kinase (DNA-PKcs) associates with Ku70/80 at the break, auto-phosphorylates, and phosphorylates other factors to facilitate NHEJ. To further confirm the effect of E7 on cNHEJ in the setting of IR mediated DNA damage with additional assays, we assessed the formation of pDNA-PKcs foci following IR at early time points when NHEJ is uniquely used. In a U2OS cell line stably transfected with an HPV16 E7 expressing vector resulted in significant downregulation of pRB (FIG. 2 D) compared to vector control. Following 6 Gy of IR, the number of pDNA-PKcs foci per cell detected was significantly decreased in the E7 expressing cell line compared to empty vector (FIG. 3 A-C), supporting the notion that HPV16 E7 suppresses cNHEJ following IR-induced DNA damage.

At a Site-Directed DSB at an Endogenous Locus, HPV16 E7 Promotes Both MMEJ and HR at the Expense of cNHEJ Events.

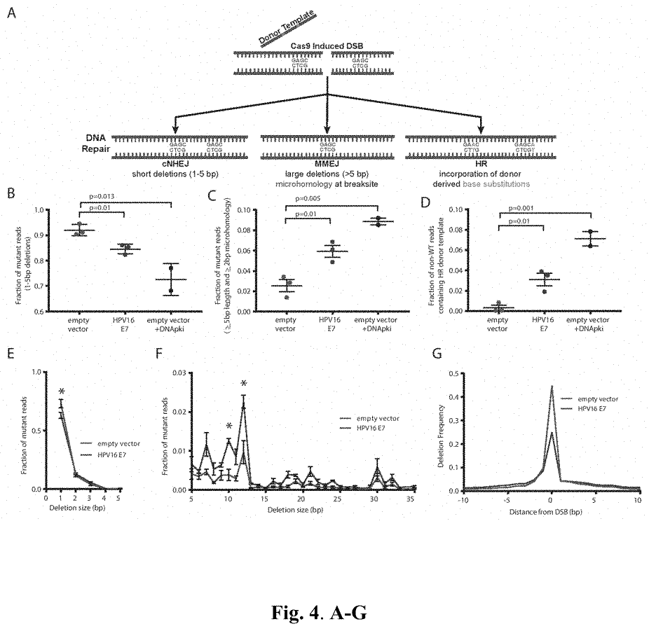

To measure DSB repair pathway tradeoffs within an endogenous genomic locus, we used the Cas9 endonuclease to create a site-directed DSB at the AAVS1 locus, chosen because of its characterization as a “safe” genomic locus for integration. Cas9 creates a blunt-ended DSB and repair events at the breaksite can be cataloged in a pool of cells using PCR amplification of a region surrounding the breaksite and massively parallel sequencing to quantify repair outcomes. To measure HR, we used a donor template containing three substitutions to reflect use of this pathway. NHEJ is measured with sequencing events with small (1-5) base pair deletions whereas MMEJ is quantified using sequencing events with large deletions and microhomology at the breaksite. All three pathways are thereby quantifiable in a single set of sequencing data in a single pool of cells (FIG. 4 A).

As controls, the DNA-PKcs inhibitor Nu7441 was used in cells to show a decrease in small deletions (1-5 base pairs), reflective of NHEJ, an increase in larger deletions (>5 bp) with microhomology (≥2 bp), and an increase in HR (FIG. 4 B-D). Similar to the DNA-PKcs inhibitor, the E7 oncoprotein suppresses NHEJ, increasing both MMEJ and HR. When the profile of deletions sizes was examined, E7 suppressed a 12 bp deletion read without microhomology most significantly (FIG. 4 E) and increased a 12 bp deletion with 5 bp of microhomology (FIG. 4 F), corresponding to MMEJ. The distribution of deleted bases surrounding the breaksite was broadened away from the breaksite in the E7-expressed cells relative to the empty vector controls (FIG. 4 G). Thus, three independent assays—cassette reporter assays, pDNApk foci, and comprehensive measurement of repair events at a site-directed break—each found a shift in repair events away from NHEJ analogous to the effect of a DNApkcs inhibitor.

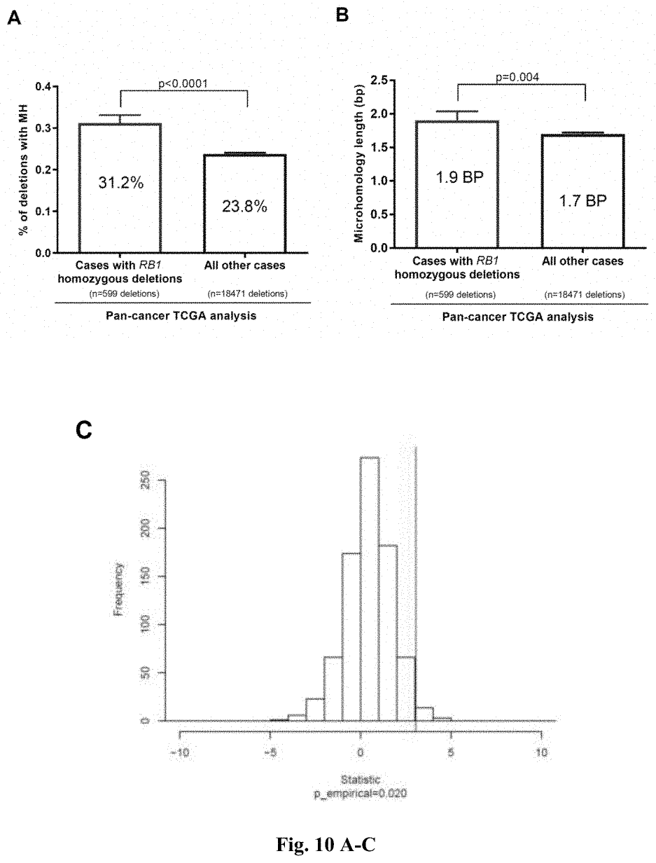

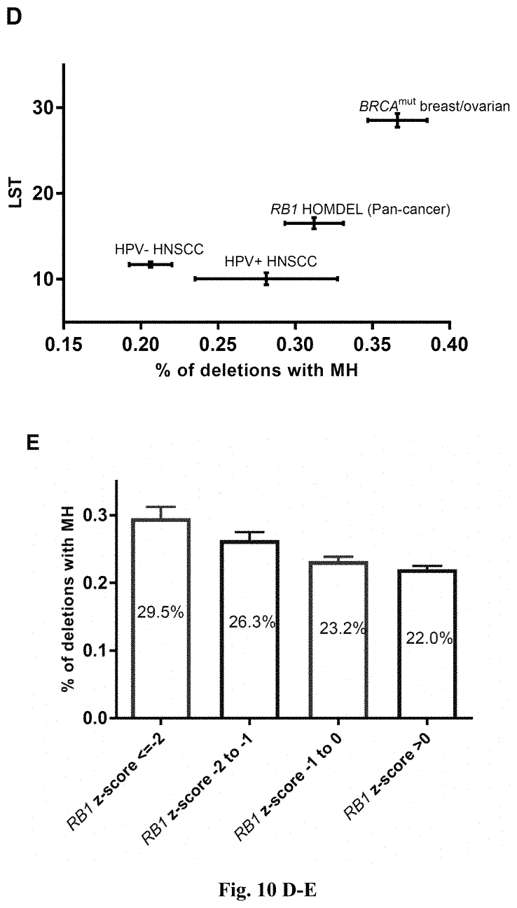

Genomic DSB repair signatures of RB1 loss. As we identified pRB downregulation in response to E7 expression as a driver of increased MMEJ and decreased cNHEJ, we assessed DNA DSB repair signatures in TCGA cases with loss of RB1 across all cancer types. Tumors with deep deletion copy number alterations (HOMDEL) of RB1 (n=303 tumors) also demonstrated a substantial increase in microhomology at deletion breakpoints (FIG. 10 A-B). RB1 loss was also associated with a small but significant increase in LST score and signature 3 contribution (FIG. 10 D). A simulation identified RB1 to be significantly associated with increased utilization of microhomology (p=0.02) compared to loss of a set of 1000 other random control genes (FIG. 10 C). Furthermore, across all cancer types, decreasing expression of pRB was associated with a progressive increase in microhomology utilization at deletion breakpoints (FIG. 10 E). Taken together, these data provide evidence that RB1 loss or suppression, outside of an effect of HPV, may also be associated with decreased utilization of cNHEJ and increased MMEJ.

### Discussion

HPV positivity represents a biomarker of paramount clinical support as SCCs of the oropharynx (3), cervix(34), vulva and anal canal(35, 36) each are more radiosensitive than their non-HPV SCC counterparts. Thus, understanding the mechanism behind HPV-associated radiosensitivity provides insight with broad implications.

There have been multiple purported mechanisms that are thought to underlie the radiosensitivity of HPV-associated tumors. The intrinsic cellular radiosensitivity that has been observed in HPV-positive HNSCC cell lines compared to HPV-negative cell lines(6) suggests that differences in the DNA damage response may be responsible, as opposed to microenvironmental or immune related mechanisms. HPV-positive HNSCCs have been found to be deficient in TGF-β signaling which has been shown to be associated with suppression of HR and an increase in alternative end-joining(37). Despite the suppression of TP53 by E6, radiation-induced activation of the remaining low level of p53 has been implicated in radiation sensitivity of HPV positive cell lines(6). Finally, E6 has also been reported to impair HR while E7 had no effect(38) and non-isogenic comparisons of 1 HPV− and 2 HPV+HNSCC cell lines have suggested HPV-associated defects in NHEJ(39) or HR(39, 40) or both(39). However, when larger cell line panels of HPV+(n=6) and HPV−HNSCC (n=5) cell lines were compared, no difference in cisplatin sensitivity has been noted, which is a surrogate for HR efficiency.(41) To deconvolve these observations on double strand break repair, we measured all three major double strand break repair pathways (NHEJ, HR, and MMEJ) in three separate assay systems and found consistent E7-mediated suppression of NHEJ and promotion of MMEJ, which matches human-level evidence in HPV-associated cancer genomes.

Abnormal end joining, thought to be mediated by MMEJ, has previously been reported in HPV immortalized and HNSCC cell lines(42) and E7 specifically has been linked to effects on DSB repair(43). Like previous reports, we found that E7 alone is profoundly radiosensitizing and is the dominant factor behind DNA DSB repair pathway deficits, namely a 30-50% suppression of NHEJ events. These missing NHEJ repair events are transferred to both increased MMEJ and increased HR events. These pathway shifts were not explained by cell cycle distribution shifts. The E7-induced pathway shifts from NHEJ to error-prone MMEJ are similar to what is observed with DNA-PKcs inhibition, which is radiosensitizing in cell line and animal models, and thus could also explain the radiosensitivity of HPV-associated squamous cell carcinomas.

As E6/E7 expression are early events in carcinogenesis of HPV-associated cancers, the subsequent effects of these genes can be measured in the pattern of deletions in cancer genomes. In HPV-associated HNSCC genomes, we find an over-representation of microhomology at deletion sites compared to HPV-negative HNSCC, indicating an overreliance of MMEJ. However, we did not find other genomic signatures of HR deficiency such as the Stratton 3 substitution pattern or the LST rearrangement score. These observations support our preclinical observations of an increased use of MMEJ that appears to result remarkably from a reciprocal response to NHEJ suppression as opposed to HRD.

The etiologic reason why the human papillomavirus suppresses NHEJ is unknown. It could be a byproduct of degradation of Rb which is described to have a direct role in NHEJ by mediating recruitment of Ku complexes to DSBs.(17) However, it could also be that increased usage of MMEJ promotes viral integration and there is a reported abundance of microhomology at sites of tumor-HPV viral genome interfaces.(18, 19) In addition, the Rb-E2F interaction reduces expression of key HR and MMEJ genes in senescent GO cells, an effect that is reversed by HPV16 E7(44). We find that an E7 mutant deficient in Rb binding does not lead to suppression of NHEJ or promotion of MMEJ, indicating that the mechanism likely involves Rb directly.

These findings have important clinical implications for the management of HPV-associated and other cancers. Biomarkers of NHEJ suppression or MMEJ overutilization may assist in personalization of therapy by characterizing sensitivity of tumors to IR or other DNA damaging agents for de-escalation or escalation of current treatments (i.e. chemoradiation treatment for HPV+HNSCC and urogenital cancers). In addition, this work provides a rationale for exploration of pharmacologic inhibition of MMEJ factors in HPV-associated cancers.

### Materials & Methods

All primers, plasmids and reagents used in this study are cataloged in Tables 1 & 3. Routine methods regarding cell culture, transfection, western blots, quantitative PCR, cell growth, cassette reporter assays, pDNA-PKcs immunofluorescence and clonogenic survival assays are described below.

### Genomic Analyses of Homologous Recombination Deficiency and Microhomology Analyses.

LST and signature 3 were calculated as per methods previously reported(22). To determine microhomology at deletion sites, deletions annotated within the TCGA .maf files were analyzed using a custom algorithm following the steps outlined in FIG. 5. Additional information is noted below.

### Cas9 Assay

To quantify NHEJ, MMEJ and HR at an endogenous breaksite, a Cas9 and gRNA expression plasmid and a donor template oligonucleotide was transfected into U2OS cells with and without E7 expressed. A PCR product surrounding the breaksite was sequenced using Illumina-based NGS technology and the pattern of deletions at the breaksite was analyzed and quantified according to methods described within the Example 2 (SI appendix).

### Example 2

### Additional Materials & Methods

Note that the references cited in Example 2 are provided in Reference List No. 2

### Cell Lines and Culture

The DR-GFP, EJ5-GFP, and EJ2-GFP U20S cell lines were cultured in DMEM, 10% bovine growth serum, and 2.5 mM L-glutamine. Stably transfected U20S cells were created with transfection of cmv 16 E7 (Addgene plasmid #13686) and empty vector backbone pCMV bam neo (Addgene plasmid #16440). Stable selection was conducted in G418 containing media (400 g/ml).

### Western Blot

The mouse anti-human Rb primary antibody (554136, BD Pharmingen) was used at 1:500 dilution in 5% milk, followed by goat anti-mouse IgG secondary antibody at 1:10,000 dilution (A28177, ThermoFisher Scientific).

### Quantitative RT-PCR

For confirmation of HPV16 E7 mRNA transcription, whole RNA was extracted with the RNeasy Mini Kit (Qiagen) followed by reverse transcription with iScript™ cDNA Synthesis Kit (Bio-rad). The following primers were used in an RT-PCR reaction using iQ SYBR green supermix (Biorad, Hercules, Calif.) as recommended by the supplier:

TABLE 4

| | Forward (5′ to 3′) | Reverse (5′ to 3′) |
|---|---|---|
| E1 | ATTTGAAAGCGAAGACAGCGG | TGGCGCCCTTCTACCTGTAA |
| | (SEQ ID NO. 1) | (SEQ ID NO. 2) |
| E2 | GTGGAAGTGCAGTTTGATGGAG | TCAACTTGACCCTCTACCACAG |
| | (SEQ ID NO. 3) | (SEQ ID NO. 4) |
| E5 | CAGATTACGCTGCTCGAGGT | AGACAAAAGCAGCGGACGTA |
| | (SEQ ID NO. 5) | (SEQ ID NO. 6) |
| E6 | ACAGCTGGGTTTCTCTACGTG | CAGTTACTGCGACGTGAGGT |
| | (SEQ ID NO. 7) | (SEQ ID NO. 8) |
| E6 | CGGTCGATGTATGTCTTGTT | CTGGGTTTCTCTACGTGTTC |
| | (SEQ ID NO. 9) | (SEQ ID NO. 10) |

All reactions were performed in triplicate on the Bio-rad iQ5 multicolor real-time PCR detection system with a GAPH or j-actin amplicon as the internal control.

### Cell Growth

Cell growth was analyzed using cellTiter-Glo luminescent cell viability assay (Promega USA) according to the manufacturer's protocol. HPV-E7- and empty vector-transfected U20S cells were seeded into 96-well plates with 3×103 cells per 100 μL media in triplicate. At the appropriate time (24, 48, 96 hours), each well was incubated with 100 μL cellTiter-Glo reagent at room temperature for 10 mins, then the stabilized luminescence signal was recorded by luminometer. The wells containing medium without cells was used to obtain a value for background luminescence.

### Clonogenic Survival Assays

Stable U20S-E7 and U20S-EV cell lines were seeded into 6-well plates. After 48 hours, cells were treated with 0 Gy, 2 Gy or 4 Gy of ionizing radiation. After 4 h cells were reseeded at various densities of cells per well of a 6-well plate. Colonies were fixed with 4% glutaraldehyde and then stained with 0.1% crystal violet 14 days later and counted.

### Genomic Analyses of Homologous Recombination Deficiency and Microhomology Analyses

LST(4-6) and signature 3(7) were calculated as per methods previously reported(8). As positive controls, breast and ovarian adenocarcinoma cases with bi-allelic pathogenic BRCA1 and BRCA2 mutations were identified amongst TCGA cases as previously published and compared to all other breast and ovarian adenocarcinoma cases without such mutations (N=1487).(8) HPV positive HNSCC cases were defined as those cases demonstrating E6 and E7 mRNA expression by RNA-seq as previously defined(9) and were compared to all other available TCGA HNSCC cases (N=510). LST(4-6) and signature 3(7) were calculated as per methods previously reported(8). Briefly, the somatic mutation calls were downloaded in the form of mutation annotation files (.maf) from the Broad firehose and used to determine the contribution from signature 3. Affymetrix SNP Array 6.0 array data was downloaded and used to determine LST. To determine microhomology at deletion sites, deletions annotated within the maf files were analyzed using a custom algorithm following the steps outlined in FIG. 5. The upstream sequence 5′ to deletion were compared for identity to the deleted sequence, followed by a comparison between the sequence 3′ to the deletion and the deleted sequence. The larger of the two stretches of identity was recorded as the number of base pairs of microhomology. To determine whether differences in somatic mutations between HPV+ and HPV− tumors may be accounting for differences in MMEJ, we assessed whether mutations in PIK3CA or TRAF3, two genes that are mutated at a significantly higher rate in HPV+HNSCC, were associated with MMEJ. We found no significant difference between the proportion of deletions with MH based on PIK3CA (22.9% non-mutated, 19.2% mutated, p=0.35) or TRAF3 mutation (no deletions with 3+bp of MH in HPV+ cases). Additionally, only 2 HPV+HNSCC cases were found to harbor mutations in any of the DNA repair genes within the TCGA DNA Damage Response Geneset.

Therefore, it is unlikely that direct mutations in DNA repair genes caused repair defects that in turn accounts for the increased usage of MH.

### DNA Repair Cassette Reporter Assays

The EJ2-GFP cassette involves a single I-Sce1 cut site with 8 base pairs of flanking microhomology, which if utilized for MMEJ repair, creates an intact GFP reading frame. The EJ5-GFP cassette involves two I-Sce1 sites and if NHEJ repair rejoins the two breaksites with excision of the intervening segment, then GFP expression is restored. Expression of a hybrid ISce1 endonuclease and Trex2 exonuclease in this system leaves blunt ends which are preferentially repaired by NHEJ(10). Finally, the DR-GFP cassette involves two sequential defective GFP sequences. A I-Sce1 cut site is in the first GFP and if HR is used with the sister chromatid, the sequence defect can be repaired to a full GFP expression sequence. U2OS cell lines with integrated double strand break repair reporter systems (EJ2-GFP, EJ5-GFP, DR-GFP)(1) were transiently co-transfected with I-Sce1 restriction enzyme and plasmids expressing individual HPV16 oncoproteins (E1, E2, E5, E6, E7) or a HPV16 mutant defective in pRB binding (E7 C24G)(2, 11) (Addgene numbers 10859, 24123, 37874, 8642, 13686, 13692). For the EJ5-GFP system, to measure cNHEJ events, blunt ended DSBs were formed using an Isce1-Trex2 fusion protein expression plasmid (Addgene #44024)(12). U2OS cells were plated in 6 well plates at 37 degrees. Twenty four hours later, 1.25 μg of Isce1 or Isce1-Trex2 restriction enzyme and 1.25 μg of HPV oncoprotein expression plasmid were transfected using Lipofectamine 3000 (Thermofisher). Cells were incubated for 72 hours, trypsinized and the percentage of GFP positive cells was measured with flow cytometry. GFP positivity was normalized to the empty vector control in each system.(10) FlowJo software was used for gating and analysis.

pDNA-PKcs Immunofluorescence

U20S cells stably transfected with an empty vector plasmid or a plasmid expressing the E7 protein were plated overnight in 4-well chamber slides. Cells were treated with 6 Gy of irradiation and cells were fixed, permeabilized, and blocked at various time points, 0 min, 30 min, 1 hr, 2 hr, 4 hr post IR. Cells were stained using the following primary antibody: anti-phospho DNA-PKcs (ab 18192) from Abcam. The secondary antibody AlexaFluor 488-labeled goat antirabbit IgG (Invitrogen) was used. Cells were lastly mounted to cover slides using Vectashield (Vector Laboratories), mounting medium with DAPI for nuclear fluorescence. Images were obtained using a Zeiss LSM 510 Meta scanning confocal microscope and were analyzed using ImageJ software. At least 750 nuclei were counted per experiment.

### Cas9 Assay

U20S cell lines stably transfected with empty vector or HPV16 E7 expression plasmid were transfected with a Cas9 and gRNA expressing plasmid targeting the AAVS1 locus. CMV 17 E7 was a gift from Karl Munger(2) (Addgene plasmid #13686) and AAVS1 T2 CRISPR in pX330 was a gift from Masato Kanemaki(13) (Addgene plasmid #72833). 72 hours following transfection, cells were harvested and genomic DNA was extracted with DNeasy (Qiagen #69506). To assess homologous directed repair, a donor template oligonucleotide was transfected with three substitutions. Then 500 ng of genomic DNA was used in a PCR reaction involving primers surrounding the expected breaksite (Forward Primer: CTCCAGGGATCCTGTGTCC (SEQ ID NO: 11) and Reverse Primer: ACAGGAGGTGGGGGTTAGAC (SEQ ID NO: 12)). Then the PCR product was purified through the Monarch PCR Clean-up Kit (#T1030S) and purified further using the E-Gel electrophoresis system (ThermoFisher Scientific). After quality control, the product was submitted for Illumina MiSeq sequencing. Paired-end libraries were sequenced. A minimum of 70,000 reads were obtained per sample. If one sample had multiple sequencing runs, they were merged by concatenating the .fastq files. The two reads in each pair were stitched together into one read using software PEAR with a minimum overlap of 10 nucleotides(14). Each stitched read was locally aligned to the wild-type sequence using substitution matrix BLOSUM62, a gap-open penalty of −10 and a gap-extension penalty of −1. Nucleotide substitutions, insertions and deletions were identified for each stitched read from the alignment with the lowest E-value, i.e. probability of occurring by chance. If there was a deletion spanning the break site, sequence match between the deleted region and the flanking region on either side of the deletion was searched, and if the sequence match was >2 bp, it is identified as microhomology. DNA repair mechanism was inferred from the alignments as follows: (i) canonical non-homology end-joining if there a deletion of <5 bp spanning the break site, (ii) microhomology-mediated end-joining if there was a deletion of >5 bp spanning the break site and a microhomology of >2 bp, or (iii) homologous recombination if the three 1-bp substitutions on the donor sequence were both detected.

### Statistics

Comparisons between experimental groups for TCGA analyses (Stratton signature 3, LST scores, microhomology), GFP-based reporters, immunofluorescence, clonogenic survival assays, and Cas-9 experiments were performed using a two-tailed student's t-test. For all statistical analyses, a two-sided p-value of <0.05 was considered to be statistically significant. Statistical analyses were performed using Prism 7.0e (GraphPad Software, Inc.) or SPSS 25.0 (IBM).

### Data Availability

The code used to define microhomology at breaksites is available at http://www.github.com/xqrongm/delmh. The .maf files used were downloaded from the Genomic Data Commons Data Portal (https://portal.gdc.cancer.gov/).

TABLE 5

| Plasmid | | | Addgene | |
|---|---|---|---|---|
| Name | Feature | Promoter | No. | Reference |
| cmv 16 E7 | HPV16 E7 | CMV | 13686 | (2) |
| | expression | | | |
| pCMV bam | Empty vector | CMV | 16440 | (3) |
| neo | backbone | | | |
| | plasmid | | | |
| P3692 | HPV16 E1 | CMV | 10859 | (15) |
| | expression | | | |
| P3662 | HPV16 E2 | CMV | 22457 | (15) |
| | expression | | | |
| MSCV-N | HPV16 E5 | PKG | 37874 | (11) |
| 16E5 | expression | | | |
| pB-actin 16 | HPV16 E6 | Human | 13713 | (16) |
| E6 | expression | β-actin | | |
| cmv 16 E7 | HPV16 E7 C24G | cmv | 13692 | |
| C24G | expression | | | |
| P1321 | HPV16 E6/E7 | Human | 8641 | (16) |
| | expression | β-actin | | |
| pCAGGS-I- | I-Sce1-Trex2 | pCAGGS | 44024 | (12) |
| Sce1-Trex2 | expression | | | |
| pCMV-I-Sce1 | I-Sce1 expression | CMV | — | (17) |
| AAVS1 in T2 | Cas9 and gRNA | U6, CMV | 72833 | (13) |
| CRISPR in | targeting AAVS1 | | | |
| pX330 | locus | | | |
| EJ2-GFP | (Integrated | pCAGGS | 44025 | (18) |
| | MMEJ reporter | | | |
| | cassette) | | | |
| EJ5-GFP | (Integrated | pCAGGS | 44026 | (18) |
| | NHEJ reporter | | | |
| | cassette) | | | |
| DR-GFP | (Integrated | CMV | 26476 | (19, 20) |
| | HR reporter | | | |
| | cassette) | | | |

TABLE 6

| Reagent | Source | Identifier |
|---|---|---|
| DMEM | MSKCC core facility | N/A |
| Bovine growth serum | ThermoFisher Scientific | SH3054103 |
| G418, Geneticin | ThermoFisher Scientific | 10131035 |
| Lipofectamine 3000 | ThermoFisher Scientific | L3000001 |
| Mouse anti-human Rb antibody | BD Pharmingen | 554136 |
| Goat anti-mouse IgG (H + L) | ThermoFisher Scientific | A28177 |
| secondary antibody, HRP | | |
| RNeasy mini kit | Qiagen | 74104 |
| iScript ™ cDNA synthesis kit | Biorad | 1708890 |
| iQ SYBR green supermix | Biorad | 1708880 |
| CellTiter-Glo | Promega | G7570 |
| Anti-phospho DNA-PKcs | Abcam | Ab18192 |
| Goat anti-rabbit IgG (H + L) | ThermoFisher Scientific | A-11008 |
| cross-adsorbed secondary | | |
| antibody, Alexa Fluor 488 | | |
| Vectashield with DAPI | Vector Laboratories | H-1200 |
| DNeasy | Qiagen | 69506 |
| Monarch PCR clean-up Kit | New England Biolabs | #T1030S |

### Example 3

The Pan-Cancer Analysis of Whole Genomes (PCAWG) project has identified characteristic deletion signature patterns (labeled ID1 through ID17 (see Alexandrov et al. “The repertoire of mutational signatures in human cancer.” Nature 578, 94-101 (2020). https://doi.org/10.1038/s41586-020-1943-3)). Of these, ID6 and ID8 are associated with cancers with biallelic inactivation of the homologous recombination genes BRCA1 and BRCA2. Thus, 1D6 and 1D8 are additional biomarkers of homologous recombination deficiency.

The ID6 signature is associated with deletions >5 bp and >2 bp of microhomology used in repairing the deletion. Thus, the ID6 signature is highly reflective of the pathway known as alternative end-joining (Alt-EJ) which is also called microhomology-mediated end-joining. Of note, the ID6 signature is a fundamentally different from other HR signatures such as base substitution signature 3(SBS3) or the large-scale transition (LST) score, which reflect base substitutions and large structure rearrangements, respectively. The ID6 signature includes short deletions, of the kind produced by Alt-EJ/MMEJ.

To evaluate for cancers that may have a pattern of high ID6 (ALT-EJ/MMEJ), but low/normal SBS3 we interrogated the PCAWG project. Radiosensitive HPV-associated tumors were previously found to exhibit elevated microhomology at deletion sites, but low/normal SBS3 and LST in The Cancer Genome Atlas (TCGA) exome datasets. This pattern correlates with elevated Alt-EJ/MMEJ as a result of suppressed NHEJ rather than HR deficiency. NHEJ is the dominant pathway responsible for radiosensitivity and thus biomarkers may be clinically useful.

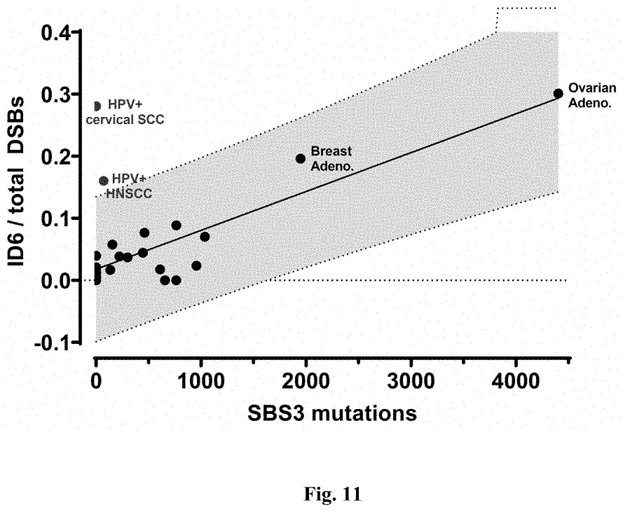

Just as in the TCGA project, we find that HPV positive tumors exhibit increased ID6 and low/normal SBS3. When correlating ID6 and SBS3, we find that the HPV-associated cancers are outside the confidence interval of the correlation, supporting a statistically significant phenotype separate from other cancers. These confirmatory data support the concept of a radiosensitivity phenotype composed of high Alt-EJ/MMEJ events and low/normal HR deficiency signatures (LST and SBS3). The results of this analysis are shown in FIG. 11.

### REFERENCE LIST NO. 1 (CITED IN BACKGROUND, DESCRIPTION OF INVENTION & EXAMPLE 1)

1. Van Dyne E A, et al. (2018) Trends in Human Papillomavirus-Associated Cancers—United States, 1999-2015. MMWR Morb Mortal Wkly Rep 67(33):918-924.
2. Chaturvedi A K (2012) Epidemiology and clinical aspects of HPV in head and neck cancers. Head Neck Pathol 6 Suppl 1:S16-24.
3. Ang K K, et al. (2010) Human papillomavirus and survival of patients with oropharyngeal cancer. N Engl J Med 363(1):24-35.
4. Leeman J E, et al. (2017) Patterns of Treatment Failure and Postrecurrence Outcomes Among Patients With Locally Advanced Head and Neck Squamous Cell Carcinoma After Chemoradiotherapy Using Modern Radiation Techniques. JAMA Oncol 3(11):1487-1494.
5. Rieckmann T, et al. (2013) HNSCC cell lines positive for HPV and p16 possess higher cellular radiosensitivity due to an impaired DSB repair capacity. Radiother Oncol 107(2):242-246.
6. Kimple R J, et al. (2013) Enhanced radiation sensitivity in HPV-positive head and neck cancer. Cancer Res 73(15):4791-4800.
7. Kotter A, et al. (2014) Inhibition of PARP1-dependent end-joining contributes to Olaparib-mediated radiosensitization in tumor cells. Mol Oncol 8(8):1616-1625.
8. Goff J P, et al. (2009) Lack of DNA polymerase theta (POLQ) radiosensitizes bone marrow stromal cells in vitro and increases reticulocyte micronuclei after total-body irradiation. Radiat Res 172(2):165-174.
9. Frit P, Barboule N, Yuan Y, Gomez D, & Calsou P (2014) Alternative end-joining pathway(s): bricolage at DNA breaks. DNA Repair (Amst) 17:81-97.
10. Ceccaldi R, et al. (2015) Homologous-recombination-deficient tumours are dependent on Poltheta-mediated repair. Nature 518(7538):258-262.
11. Bentley J, et al. (2009) Papillary and muscle invasive bladder tumors with distinct genomic stability profiles have different DNA repair fidelity and KU DNA-binding activities. Genes Chromosomes Cancer 48(4):310-321.
12. Bentley J, Diggle C P, Harnden P, Knowles M A, & Kiltie A E (2004) DNA double strand break repair in human bladder cancer is error prone and involves microhomology-associated end-joining. Nucleic Acids Res 32(17):5249-5259.
13. Gaymes T J, Mufti G J, & Rassool F V (2002) Myeloid leukemias have increased activity of the nonhomologous end-joining pathway and concomitant DNA misrepair that is dependent on the Ku70/86 heterodimer. Cancer Res 62(10):2791-2797.
14. Newman E A, et al. (2015) Alternative NHEJ Pathway Components Are Therapeutic Targets in High-Risk Neuroblastoma. Mol Cancer Res 13(3):470-482.
15. McCormick A, et al. (2017) Ovarian Cancers Harbor Defects in Nonhomologous End Joining Resulting in Resistance to Rucaparib. Clin Cancer Res 23(8):2050-2060.
16. Polkinghorn W R, et al. (2013) Androgen receptor signaling regulates DNA repair in prostate cancers. Cancer Discov 3(11):1245-1253.
17. Cook R, et al. (2015) Direct involvement of retinoblastoma family proteins in DNA repair by non-homologous end-joining. Cell Rep 10(12):2006-2018.
18. Parfenov M, et al. (2014) Characterization of HPV and host genome interactions in primary head and neck cancers. Proc Natl Acad Sci USA 111(43):15544-15549.
19. Hu Z, et al. (2015) Genome-wide profiling of HPV integration in cervical cancer identifies clustered genomic hot spots and a potential microhomology-mediated integration mechanism. Nat Genet 47(2):158-163.
20. Abkevich V, et al. (2012) Patterns of genomic loss of heterozygosity predict homologous recombination repair defects in epithelial ovarian cancer. Br J Cancer 107(10):1776-1782.
21. Popova T, et al. (2012) Ploidy and large-scale genomic instability consistently identify basal-like breast carcinomas with BRCA1/2 inactivation. Cancer Res 72(21):5454-5462.
22. Riaz N, et al. (2017) Pan-cancer analysis of bi-allelic alterations in homologous recombination DNA repair genes. Nat Commun 8(1):857.
23. Mutter R W, et al. (2017) Bi-allelic alterations in DNA repair genes underpin homologous recombination DNA repair defects in breast cancer. J Pathol 242(2):165-177.
24. Manie E, et al. (2016) Genomic hallmarks of homologous recombination deficiency in invasive breast carcinomas. Int J Cancer 138(4):891-900.
25. Alexandrov L B, et al. (2013) Signatures of mutational processes in human cancer. Nature 500(7463):415-421.
26. Nik-Zainal S, et al. (2016) Landscape of somatic mutations in 560 breast cancer whole-genome sequences. Nature 534(7605):47-54.
27. Kent T, Chandramouly G, McDevitt S M, Ozdemir A Y, & Pomerantz R T (2015) Mechanism of microhomology-mediated end-joining promoted by human DNA polymerase theta. Nat Struct Mol Biol 22(3):230-237.
28. Elvin J A, Chura J, Gay L M, & Markman M (2017) Comprehensive genomic profiling (CGP) of ovarian clear cell carcinomas (OCCC) identifies clinically relevant genomic alterations (CRGA) and targeted therapy options. Gynecol Oncol Rep 20:62-66.
29. Kawamura K, et al. (2004) DNA polymerase theta is preferentially expressed in lymphoid tissues and upregulated in human cancers. Int J Cancer 109(1):9-16.
30. Seki M, Marini F, & Wood R D (2003) POLQ (Pol theta), a DNA polymerase and DNA-dependent ATPase in human cells. Nucleic Acids Res 31(21):6117-6126.
31. Bennardo N, Cheng A, Huang N, & Stark J M (2008) Alternative-NHEJ is a mechanistically distinct pathway of mammalian chromosome break repair. PLoS Genet 4(6):e1000110.
32. Demers G W, Espling E, Harry J B, Etscheid B G, & Galloway D A (1996) Abrogation of growth arrest signals by human papillomavirus type 16 E7 is mediated by sequences required for transformation. J Virol 70(10):6862-6869.
33. Chakravarthy A, et al. (2016) Human Papillomavirus Drives Tumor Development Throughout the Head and Neck: Improved Prognosis Is Associated With an Immune Response Largely Restricted to the Oropharynx. J Clin Oncol 34(34):4132-4141.
34. Rodriguez-Carunchio L, et al. (2015) HPV-negative carcinoma of the uterine cervix: a distinct type of cervical cancer with poor prognosis. BJOG 122(1):119-127.
35. Mai S, et al. (2015) Prognostic Relevance of HPV Infection and p16 Overexpression in Squamous Cell Anal Cancer. Int J Radiat Oncol Biol Phys 93(4):819-827.
36. Meulendijks D, et al. (2015) HPV-negative squamous cell carcinoma of the anal canal is unresponsive to standard treatment and frequently carries disruptive mutations in TP53. Br J Cancer 112(8):1358-1366.
37. Liu Q, et al. (2018) Subjugation of TGFbeta Signaling by Human Papilloma Virus in Head and Neck Squamous Cell Carcinoma Shifts DNA Repair from Homologous Recombination to Alternative End Joining. Clin Cancer Res 24(23):6001-6014.
38. Wallace N A, et al. (2017) High-Risk Alphapapillomavirus Oncogenes Impair the Homologous Recombination Pathway. J Virol 91(20).
39. Weaver A N, et al. (2015) DNA double strand break repair defect and sensitivity to poly ADP-ribose polymerase (PARP) inhibition in human papillomavirus 16-positive head and neck squamous cell carcinoma. Oncotarget 6(29):26995-27007.
40. Dok R, et al. (2014) p16INK4a impairs homologous recombination-mediated DNA repair in human papillomavirus-positive head and neck tumors. Cancer Res 74(6):1739-1751.
41. Busch C J, et al. (2016) Similar cisplatin sensitivity of HPV-positive and -negative HNSCC cell lines. Oncotarget 7(24):35832-35842.
42. Shin K H, et al. (2006) Abnormal DNA end-joining activity in human head and neck cancer. Int J Mol Med 17(5):917-924.
43. Park J W, et al. (2014) Human papillomavirus type 16 E7 oncoprotein causes a delay in repair of DNA damage. Radiother Oncol 113(3):337-344.
44. Collin G, Huna A, Warnier M, Flaman J M, & Bernard D (2018) Transcriptional repression of DNA repair genes is a hallmark and a cause of cellular senescence. Cell Death Dis 9(3):259.

### REFERENCE LIST NO. 2 (CITED IN EXAMPLE 2)

1. Howard S M, Yanez D A, & Stark J M (2015) DNA damage response factors from diverse pathways, including DNA crosslink repair, mediate alternative end joining. PLoS Genet 11(1):e1004943.
2. Gonzalez S L, Stremlau M, He X, Basile J R, & Munger K (2001) Degradation of the retinoblastoma tumor suppressor by the human papillomavirus type 16 E7 oncoprotein is important for functional inactivation and is separable from proteasomal degradation of E7. J Virol 75(16):7583-7591.
3. Baker S J, Markowitz S, Fearon E R, Willson J K, & Vogelstein B (1990) Suppression of human colorectal carcinoma cell growth by wild-type p53. Science 249(4971):912-915.
4. Popova T, et al. (2012) Ploidy and large-scale genomic instability consistently identify basal-like breast carcinomas with BRCA1/2 inactivation. Cancer Res 72(21):5454-5462.
5. Birkbak N J, et al. (2012) Telomeric allelic imbalance indicates defective DNA repair and sensitivity to DNA-damaging agents. Cancer Discov 2(4):366-375.
6. Abkevich V, et al. (2012) Patterns of genomic loss of heterozygosity predict homologous recombination repair defects in epithelial ovarian cancer. Br J Cancer 107(10):1776-1782.
7. Alexandrov L B, et al. (2013) Signatures of mutational processes in human cancer. Nature 500(7463):415-421.
8. Riaz N, et al. (2017) Pan-cancer analysis of bi-allelic alterations in homologous recombination DNA repair genes. Nat Commun 8(1):857.
9. Cancer Genome Atlas N (2015) Comprehensive genomic characterization of head and neck squamous cell carcinomas. Nature 517(7536):576-582.
10. Gunn A & Stark J M (2012) I-SceI-based assays to examine distinct repair outcomes of mammalian chromosomal double strand breaks. Methods Mol Biol 920:379-391.
11. Rozenblatt-Rosen 0, et al. (2012) Interpreting cancer genomes using systematic host networkperturbations by tumour virus proteins. Nature 487(7408):491-495.
12. Gunn A, Bennardo N, Cheng A, & Stark J M (2011) Correct end use during end joining of multiple chromosomal double strand breaks is influenced by repair protein RAD50, DNA-dependent protein kinase DNA-PKcs, and transcription context. J Biol Chem 286(49):42470-42482.
13. Natsume T, Kiyomitsu T, Saga Y, & Kanemaki M T (2016) Rapid Protein Depletion in Human Cells by Auxin-Inducible Degron Tagging with Short Homology Donors. Cell Rep 15(1):210-218.
14. Zhang J, Kobert K, Flouri T, & Stamatakis A (2014) PEAR: a fast and accurate Illumina Paired-End reAd mergeR. Bioinformatics 30(5):614-620.
15. Sakai H, Yasugi T, Benson J D, Dowhanick J J, & Howley P M (1996) Targeted mutagenesis of the human papillomavirus type 16 E2 transactivation domain reveals separable transcriptional activation and DNA replication functions. J Virol 70(3):1602-1611.
16. Munger K, Phelps W C, Bubb V, Howley P M, & Schlegel R (1989) The E6 and E7 genes of the human papillomavirus type 16 together are necessary and sufficient for transformation of primary human keratinocytes. J Virol 63(10):4417-4421.
17. Liang F, Han M, Romanienko P J, & Jasin M (1998) Homology-directed repair is a major doublestrand break repair pathway in mammalian cells. Proc NatlAcad Sci USA 95(9):5172-5177.
18. Bennardo N, Cheng A, Huang N, & Stark J M (2008) Alternative-NHEJ is a mechanistically distinct pathway of mammalian chromosome break repair. PLoS Genet 4(6):e1000110.
19. Weinstock D M, Nakanishi K, Helgadottir H R, & Jasin M (2006) Assaying double-strand break repair pathway choice in mammalian cells using a targeted endonuclease or the RAG recombinase. Methods Enzymol 409:524-540.
20. Pierce A J, Johnson R D, Thompson L H, & Jasin M (1999) XRCC3 promotes homology-directed repair of DNA damage in mammalian cells. Genes Dev 13(20):2633-2638.

1-46. (canceled)

47. A method of predicting the radiation sensitivity of a human papillomavirus (HPV) positive tumor in a human subject, the method comprising: (A) obtaining a sample of genomic DNA from an HPV-positive tumor in a human subject, (B) performing an assay to determine the nucleotide sequence of the genomic DNA, and (C) performing an analysis of the nucleotide sequence of the genomic DNA to identify the presence of:
(i) microhomology at DNA break-sites, and
(ii) DNA repair by homologous recombination or DNA repair by non-homologous end joining (NHEJ),
wherein the existence of both: (a) high levels of microhomology at break-sites and (b) either normal or elevated levels of DNA repair by homologous recombination or reduced levels of DNA repair by NHEJ in the nucleotide sequence of the genomic DNA, indicates that the HPV positive tumor in the subject is likely to be sensitive to treatment with radiation.

48. The method of claim 47, wherein the presence of microhomology at DNA break-sites is identified by the presence of deletions of less than 200 bp in the genome that are flanked on either side by micro-homologous sequences of at least 3 base pairs in length.

49. The method of claim 47, wherein the presence of DNA repair by homologous recombination is identified by the presence of large-scale state transitions (LST), loss of heterozygosity (LOH), telomeric allelic imbalance (TAI), or Signature 3 rearrangement signatures.

50. The method of claim 47, further comprising performing an assay to determine if the tumor in the subject is HPV-positive.

51. The method of claim 47, further comprising performing an assay to determine if the tumor in the subject expresses the HPV E7 protein.

52. The method of claim 47, wherein the analysis of the sequence comprises comparing the nucleotide sequence of the genomic DNA from the tumor in the subject to the nucleotide sequence of a control genomic DNA sequence, wherein the control genomic DNA sequence is from a radiation resistant tumor, a radiation sensitive tumor, an HPV negative tumor, an HPV positive tumor, a non-tumor cell from an individual other than the subject, or a non-tumor cell from the subject.

53. The method of claim 47, wherein the tumor is a squamous cell carcinoma (SCC).

54. The method of claim 47, wherein the tumor is a squamous cell carcinoma of the head or neck (HNSCC).

55. The method of claim 47, wherein the tumor is a squamous cell carcinoma (SCC) of the oropharynx, cervix, vulva or anal canal.

56. The method of claim 47, wherein the tumor is HPV-16, HPV-18, HPV-31, HPV-33, HPV-35, HPV-39, HPV-45, HPV-51, HPV-52, HPV-56, HPV-58 or HPV-59 positive.

57. A method of treating a human papillomavirus (HPV) positive tumor in a human subject in need thereof, the method comprising:
(a) determining that genomic DNA from an HPV-positive tumor in a human subject contains: (i) a high level of microhomology at DNA break-sites, and (ii) either a normal or elevated level of DNA repair by homologous recombination or a reduced level of DNA repair by non-homologous end joining (NHEJ), and
(b) subsequently treating the HPV-positive tumor in the human subject with radiation.

58. The method of claim 57, wherein the level of microhomology at DNA break-sites is determined by quantifying the number of deletions of less than 200 bp in the genome that are flanked on either side by micro-homologous sequences of at least 3 base pairs in length.

59. The method of claim 57, wherein the level of DNA repair by homologous recombination is determined by quantifying the number or degree of large-scale state transitions (LST), loss of heterozygosity (LOH), telomeric allelic imbalance (TAI), or Signature 3 rearrangement signatures.

60. The method of claim 57, further comprising performing an assay to determine if the tumor in the subject is HPV-positive.

61. The method of claim 57, further comprising performing an assay to determine if the tumor in the subject expresses the HPV E7 protein.

62. The method of claim 57, wherein step (a) comprises comparing the nucleotide sequence of the genomic DNA from the tumor in the subject to the nucleotide sequence of a control genomic DNA sequence, wherein the control genomic DNA sequence is from a radiation resistant tumor, a radiation sensitive tumor, an HPV negative tumor, an HPV positive tumor, a non-tumor cell from an individual other than the subject, or a non-tumor cell from the subject.

63. The method of claim 57, wherein the tumor is a squamous cell carcinoma (SCC).

64. The method of claim 57, wherein the tumor is a squamous cell carcinoma of the head or neck (HNSCC).

65. The method of claim 57, wherein the tumor is a squamous cell carcinoma (SCC) of the oropharynx, cervix, vulva or anal canal.

66. The method of claim 57, wherein the tumor is HPV-16, HPV-18, HPV-31, HPV-33, HPV-35, HPV-39, HPV-45, HPV-51, HPV-52, HPV-56, HPV-58 or HPV-59 positive.
